(19) United States
(12) Patent Application Publication
(10) Pub. No.: US 2019/0255348 A1
(43) Pub. Date: Aug. 22, 2019

(54) Apparatus and method for the treatment of melanoma

(71) Applicant: Emblation Limited, Alloa (GB)

(72) Inventors: Gary Beale, Alloa (GB); Eamon McErlean, Alloa (GB); Matthew Kidd, Alloa (GB)

(21) Appl. No.: 16/277,171

(22) Filed: Feb. 15, 2019

Related U.S. Application Data
(60) Provisional application No. 62/763,119, filed on Feb. 16, 2018.

(51) Int. Cl. A61N 5/02; C07K 16/28; A61K 31/655; A61K 31/495; A61K 47/64; A61K 31/337; A61K 33/243; A61K 31/282; A61K 31/475; A61B 18/18; A61N 5/04

(52) U.S. Cl. CPC A61N 5/025; A61B 2018/00589; C07K 16/2827; A61K 31/655; A61K 31/495; A61K 47/643; A61K 31/337; A61K 33/243; A61K 31/282; A61K 31/475; A61B 18/1815; A61N 5/04; C07K 2317/24; C07K 2317/21; C07K 16/2818

(57) ABSTRACT

Described herein are methods and apparatus for the treatment of melanoma skin cancer.

### CROSS REFERENCE TO RELATED APPLICATION

This application claims the benefit of priority under 35 U.S.C. § 119(e) to U.S. Provisional Patent Application Ser. No. 62/763,119 filed Feb. 16, 2018, which is incorporated herein by reference.

### FIELD OF THE INVENTION

The present invention provides methods and apparatus for the treatment of melanoma skin cancer.

### BACKGROUND

Melanoma is primarily a cutaneous disease, but a significant number of cases are reported each year in ocular and mucosal locations. Of the ocular melanomas, most occur in the choroid, with the ciliary body and iris being less common locations. Primary mucosal melanomas are most common in the nasal cavity, sinuses, oropharynx, rectum, vulva (including clitoris and labia), and vagina. Less commonly, primary melanomas can also arise in the oesophagus, stomach, small and large intestines, gallbladder, bile ducts, larynx, trachea, bronchi, lung, urethra, bladder, and cervix. Primary melanomas have also been reported in the prostate, salivary glands, kidney, thyroid, thymus, pancreas, ovary, and adrenal glands. With many cases likely arising in extensions of the mucosal network, the ratio of these reported cases between true primary melanomas and metastases is difficult to determine. In general, extracutaneous melanomas have a much worse prognosis than their cutaneous equivalents.

Melanoma is not a single entity but, rather, a genetically heterogeneous group of tumours, which are tied together only by the common bond of originating within a melanocyte. External factors, mutations, and epigenetic events all contribute to melanomagenesis, indeed there are multiple molecular paths to melanoma.

The MAP (mitogen-activated protein) kinase pathway is one of the main conduits for transmitting signals from the cell surface to the nucleus. More than 90% of melanomas have an activated MAP kinase pathway. Signalling through this pathway leads to activation of the cell, resulting in progression through the cell cycle and regulation of apoptosis and survival mechanisms. Cellular activation often begins with activation of upstream molecules (usually via cell surface receptors) followed by sequential activation of more downstream molecules, usually through phosphorylation. Furthermore, there are multiple members in each family of molecules, and there are elaborate checkpoints, compensatory pathways, and feedback mechanisms.

Similar to the MAP kinase pathway is the PI3K/Akt/mTOR (Phosphoinositide 3-kinase/Protein Kinase B/The mechanistic target of rapamycin) pathway that runs parallel. The PI3K/Akt/mTOR pathway is an enzymatic cascade leading to cellular growth, proliferation, and survival, with main signal propagation as follows: PI3K Akt mTOR. PTEN (Phosphatase and tensin homolog) is an inhibitor of this pathway at the point of PI3K. Most cancers, including up to 60% of melanomas, have alterations at some point within this pathway.

Melanoma models of progression from a normal melanocyte to metastatic melanoma are still in development but in general follow paths such as: melanocyte→nevus→dysplastic (atypical) nevus→melanoma in-situ→invasive melanoma→metastasis. This progression occurs with an accumulation of somatic mutations and epigenetic events, possibly mediated, in part, by UV radiation due to its mutagenic effects on DNA. Development of tumour progression models remains important for understanding the molecular players in the “growth/survival H arrest/death” balance of the cell and points of potential therapeutic interventions.

The distribution of melanomas is related to “type”; the back is the most common site for melanomas in males and the lower extremities in females. Specific sub-types of melanoma favour certain areas.

“Lentigo maligna” melanoma occurs most frequently on the face and sun-exposed upper extremities of elderly people. The “superficial spreading” melanoma may develop on any part of the body and at any age. It is particularly common on the trunk in males and the lower extremities in females.

“Nodular melanomas” have no early radial growth stage and thus they are nodular, polypoid in shape.

“Acral lentiginous melanomas” develop on palmar, plantar, and subungual skin. They are particularly common in elderly males of black, eastern Asian and Chinese populations.

“Desmoplastic melanoma” is another predominantly male type that is usually found on the head and neck region as a spreading hard plaque or a bulky, firm swelling.

Treatments

Imiquimod-medication that acts as an immune response modifier acting on several levels, which appear to synergistically bringing about the anti-tumour activity of the compound. Imiquimod signals to the innate immune system via the TLR7 (toll-like receptor 7), commonly involved in pathogen recognition. Cells activated by TLR-7 secrete cytokines (primarily IFN-α (interferon-α), IL-6 (interleukin-6), and TNF-α (tumour necrosis factor-α). It is thought that imiquimod can also trigger the adaptive immune system as when applied to skin and can activate Langerhans cells, which subsequently migrate to local lymph nodes. Other cell types activated by imiquimod include NK (natural killer cells), macrophages and B-lymphocytes.

Melanoma is primarily a surgically treated disease, and surgery remains virtually the only necessary treatment modality for thin melanomas except for lentigo maligna where cryotherapy, topical imiquimod, (either alone or in combination) and radiotherapy, have all been proposed as suitable methods of treatment. Therapies that modulate the immune system and others that target specific molecular defects within the tumour cells have brought hope for improving survival for the individual advanced melanoma patient.

Metastases

The presence of metastases indicates a poor prognosis as the 5-year survival rate is still around 5%. Metastases in cases of malignant melanoma are, in the first instance at least, usually to regional lymph nodes. The probability of nodal metastases is associated with the tumour depth and other histologic parameters. Additional primary melanomas may develop in 2-5% of patients with malignant melanoma. The sentinel lymph nodes in the lymphatic basins are the first nodes to be involved in metastases. Survival data are dependent on the stage. With early stage, IA for example, the 5-year survival is 97%, whereas the 5-year survival for stage IV (distant metastatic) disease is less than 30%. Patients with thick melanomas have a reduced risk of recurrence with time as 80% of recurrences occur within the first 3 years.

Prognosis is dependent on the age and sex of the patient with more favourable outcomes for younger and female populations. This is not true for immunosuppressed patients who overall have a significantly poorer survival. A thinner lesion also leads to a better prognosis. Conversely, the number of involved lymph nodes and size of the deposits negatively impact prognosis. A single positive lymph node (N1) defined as stage III leads to a 5-year survival of 40-70%. Stage IV disease has a uniformly poor prognosis, with a 1-year survival of 40-60%

A gradual increase of a tumour infiltrating lymphocytes during melanoma tumourigenesis reflects antigenicity of the tumour cells such as the Tregs (regulatory T cells). Tregs ensure a controlled immune response upon pathogen encounter and thereby prevent immune pathology. However, excessive suppression by Tregs can hamper pathogen clearance, promote chronic infection and crucially restrain anti-tumour immune responses thus promoting tumour progression. Accumulation of Tregs in melanoma is frequently recorded and the ratio of CD8+T (cluster of differentiation 8) cells versus Tregs in the tumour microenvironment is predictive for survival of patients with melanoma. Animal studies have shown that Tregs depletion greatly increases the efficacy of immunotherapy. Zhang et al, 2017 (Effects of microwave ablation on T-cell subsets and cytokines of patients with hepatocellular carcinoma:

Targeted Therapies

Oncogenes play an important role in the regulation or synthesis of proteins linked to tumourigenic cell growth. Drug development has focused on targeting these aberrantly activated oncoproteins. Targeted therapy has also shifted the focus away from treating tumours based on anatomic site or cell of origin towards treatment based on specific molecular events that drive oncogenesis. Such a mutation target is the FDA approved B-Raf inhibitor, vemurafenib or dabrafenib however neither offer a definitive cure with extension of life measured in months.

Almost all melanomas have an activated MAPK (mitogen-activated protein kinases) pathway, culminating in activated MEK and ERK (extracellular signal-regulated kinases), whether by mutated B-Raf or other molecules. Clinical trials using MEK inhibitor monotherapy include trametinib, selumetinib and pimasertib.

Kit inhibitors, such as imatinib mesylate have poor predicative efficacy as the therapy outcome is linked to the type of KIT (Tyrosine Kinase Inhibitor) aberration. These secondary KIT mutations are the focus of a range of compounds such as nilotinib, sunitinib malate, dasatinib, sorafenib tosylate, masatinib mesylate, and midostaurin. Pharmacologic inhibition to other molecules activated in melanoma is being investigated. Candidate molecules to target, as monotherapy or in combination regimens, include the following: N-Ras (Neuroblastoma-RAS), PI3K, Akt, mTOR and the mTOR complexes mTORC1/2.

Modification of the immune system has attained recent success as a strategy in the treatment of advanced melanoma. Mechanisms include vaccines, adoptive T cell therapy using autologous tumour-infiltrating lymphocytes, cytokine therapy, and immunomodulatory agents. The established drug, aldesleukin has been used for treating metastatic melanoma where it can induce partial responses in subsets of patients but with little effect on overall survival and is associated with significant toxicity. More effective interventions are ipilimumab and tremelimumab, monoclonal antibodies that act as an immunomodulator and are FDA approved for the treatment of advanced melanoma. The antibodies bind to CTLA-4 (cytotoxic T lymphocyte antigen 4) thereby reducing the activity of Tregs to enhance the overall immune response.

Pembrolizumab and nivolumab are also antibody directed drugs that focus on the programmed cell death 1 (PD-1) pathway. The PD-1 receptor on activated T cells normally binds to ligands on antigen-presenting cells (APCs), suppressing the natural immune response, regardless of the tumour microenvironment. Additionally, cancers in general tumour cells can be induced to express PDL-1 by cytokines such as (IFN-γ interferon-γ) in a local inflammatory state. Such immune checkpoint inhibitor antibodies interfere with this process, causing a heightened immune response with antitumour activity.

Combination immunotherapy is recommended for both first line and second line or subsequent metastatic or unresectable melanomas. Simultaneous targeting of multiple checkpoints along series or parallel circuits within malignant cells may provide a greater initial tumour response and may minimize resistance. Combinations of MAP kinase inhibitors and inhibitors of other pathways (PIK3/Akt/mTOR, p16/CDK4/Rb, etc.) are being explored.

T-Vec (Talimogene laherparepvec) is a modified herpes virus type 1 oncolytic virus therapy that uses a locally administered virus to infect and kill cancer cells while avoiding normal, healthy cells. The virus replicates within tumours and produces the immune stimulatory protein GM-CSF (Granulocyte-macrophage colony-stimulating factor) and also causes the tumour cells to lyse thereby releasing tumour-derived antigens which, along with GM-CSF stimulates an anti-tumour immune response.

Mild hyperthermia involving temperature spiking to mimic fever has been shown by Choi et al, (Am J Pathol 172:367-777, 2008) to down-regulate the PI3-K/Akt signalling pathway. Inhibition of PI3-K/Akt by heat also has an inhibitory effect on neutrophil migration which downregulates the inflammatory response.

Microwave radiation can easily penetrate deeply within the epidermal layers to the dermis. The depth of melanoma tumours featured in the historical scoring of malignancy state is known as the Clark Scale. Level 1 is also called melanoma in situ—the melanoma cells are only in the outer layer of the skin (the epidermis); Level 2 is when there are melanoma cells in the layer directly under the epidermis (the papillary dermis); Level 3, the melanoma cells are throughout the papillary dermis and touching on the next layer down (the reticular dermis); Level 4 when the melanoma has spread into the reticular or deep dermis; and Level 5 is when the melanoma has grown into the layer of fat under the skin (subcutaneous fat). Depending on the location of skin the depth p to Level 4 is 2-4 mm. Clark's level has prognostic significance only in patients with very thin (<1 mm) melanomas.

The overall Melanoma may be classed by tumour, node and metastasis (TNM) staging nomenclature. The stage of the cancer helps clinicians decide what treatment is most appropriate.

T staging: This means the melanoma cells are only in the very top layer of the skin surface T1 means the melanoma is less than 1 mm thick; T2 means the melanoma is between 1 mm and 2 mm thick; T3 means the melanoma is between 2 mm and 4 mm thick; T4 means the melanoma is more than 4 mm thick.

N staging: describing whether cancer cells are in the nearby lymph nodes or lymphatic ducts: N0 means that the nearby lymph nodes don't contain melanoma cells; N1 means there are melanoma cells in one lymph node; N2 means there are melanoma cells in 2 or 3 lymph nodes; N3 means there are melanoma cells in 4 or more lymph nodes. The N part of the stage is further divided into groups a, b and c.

Na means the cancer in the lymph node can only be seen by microscope (micrometastasis); Nb means there are obvious signs of cancer in the lymph node (macrometastasis); Nc means that there are melanoma cells in small areas of skin less than 2 cm from the primary melanoma (satellite metastases) or in the skin lymph channels (in transit metastases). The process by which cancer cells spread to distal parts of the body from the primary tumour is called metastasis. At these sites the metastatic cancer cells have features like that of the primary cancer.

Numerous clinical trials have studied hyperthermia in combination with radiation therapy and/or chemotherapy. These studies have focused on the treatment of many types of cancer, including melanoma, sarcoma and cancers of the head and neck, brain, lung, esophagus, breast, bladder, rectum, liver, appendix, cervix, and peritoneal lining (mesothelioma). Many of these studies have shown a significant reduction in tumour size when hyperthermia is combined with other treatments.

In local hyperthermia, heat is applied to a small area, such as a tumour, using various techniques that deliver energy to heat the tumour. Different types of energy may be used to apply heat, including laser, radiofrequency, and ultrasound. Microwave radiation can heat deeper layers resulting in a superior deposition of energy within the diseased region. Additionally, microwave energy causes heating and gradual desiccation of tissue without generating the harmful smoke plume associated with high energy vaporization.

It is established that heat shock proteins (HSP) are produced in response to various tissue stresses or damage resulting from physical or environmental influences. Heat shock proteins are a class of functionally related proteins whose expression is increased when cells are exposed to elevated temperatures or other stress. It has been suggested that the heat shock proteins may protect the cells from other stressors or against further damage. Heat shock proteins are also involved in antigen presentation, steroid receptor function, intracellular trafficking, nuclear receptor binding, and apoptosis. Typically exposure of cells to a heat shock temperature of 42 degrees C. results in transient activation of heat shock factor (HSF). The DNA-binding activity increases, plateaus, and dissipates, during which the intracellular levels of heat shock protein increase. Heat shock proteins can perform specific functions, for example, extracellular and membrane bound heat-shock proteins, especially HSP70 are involved in binding antigens and presenting them to the immune system.

The upregulation of the heat shock proteins is a principle part of the heat shock response and is primarily induced by the heat shock factor. Cellular stresses, such as increased temperature, can cause proteins in the cell to denature. Heat shock proteins bind to the denatured proteins and dissociate from HSF to form trimers and translocate to the cell nucleus to activate transcription resulting in the production of new heat shock proteins which bind to more denatured cells.

In research into the physiological heating effects of electromagnetic fields, high frequency microwave energy (existing between 500 MHz to 200 GHz) has been reported to thermally produce elevated levels of specific heat shock proteins in tissue for example Ogura, British Journal of Sports Medicine 41, 453-455. (2007)) teaches that HSP90, HSP72, HSP27 levels are significantly higher in heated vastus lateralis muscle compared with unheated controls. Tonomura et al. (J Orthop Res. 26(1):34-41. (2008)) teach that in vivo HSP70 expression in rabbit cartilage increases with the application of moderate levels of microwave power (20-40 W). Additionally de Pomerai et al. (Enzyme and Microbial Technology 30, 73-79 (2002)) teaches that prolonged exposure to weak microwave fields (750-1000 MHz, 0.5 W) at 25° C. induces a heat-shock response in transgenic C. elegans strains carrying HSP16 reporter genes.

In other unrelated research Shevtsov M and Multhoff G (2016) Heat Shock Protein—Peptide and HSP-Based Immunotherapies for the Treatment of Cancer Front. Immunol. 7:171 (10.3389/fimmu.2016.00171) teaches that tumours can be treated using vaccines containing heat shock proteins as immunologic adjuvants (HSPs). Amongst other pathways, the systemic antitumor immune response was found to be mediated by CD4+ and CD8+ T cells. It has been speculated that HSPs may also be involved in binding with protein fragments from dead malignant cells and highlighting them to the immune system thus boosting the effectiveness of the vaccine, e.g. Oncophage (Antigenics Inc, Lexington, Mass.).

Heat shock proteins not only carry antigens but can also induce maturation of dendritic cells, resulting in a more efficient antigen presentation. It is known that hyperthermia can promote the activation of the Langerhans cells. Langerhans cells are the dendritic cells of the skin which continuously monitor the extracellular matrix of the skin and capture, uptake and process antigens to become antigen presenting cells (APC's). Particles and antigens are carried to draining lymph nodes for presentation to T lymphocytes. T cells release chemokines which cause the skin to be infiltrated by neutrophils, resulting in a swelling response which has been observed by Gao et al. (Chin Med J (Engl), 122(17):2061-3 (2009)) to occur before resolution of a HPV infection which is particularly relevant to HPV-positive melanoma states.

It has also been suggested (Dréau et al., (2000); Human papilloma virus in melanoma biopsy specimens and its relation to melanoma progression: Ann Surg. 2000 May; 231(5):664-71) that HPV can be identified in stage III and IV melanoma. Indeed the presence of HPV is correlated with rapid melanoma progression and it is suggested that HPV may serve as a cofactor in the development of melanoma and may modulate a more aggressive phenotype in HPV-containing melanoma cells. HPV is not associated with human primary malignant melanoma in non sun-exposed body areas like mucous membranes (see Dahlgren et al; Acta Oncol: Human papilloma virus (HPV) is rarely detected in malignant melanomas of sun sheltered mucosal membranes: 2005; 44(7):694-9) this would support the cofactory argument in sun-exposed sites.

Hyperthermia also increases the expression of key adhesion molecules in secondary lymphoid tissues. Additionally, hyperthermia can also act directly on lymphocytes to improve their adhesive properties. Hyperthermia increases the intravascular display of homeostatic chemokines, and certain inflammatory chemokines which have been proposed by Skitzki et al. (Curr Opin Investig Drugs, 10(6):550-8 (2009)) to be classical HSP's based on their regulation by HSP transcription factors.

The PI3K (phosphatidylinositol 3-kinase) AKT pathway is a critical regulator of many essential cellular processes. In addition to playing an important role in normal cellular physiology, activation of PI3K-AKT signaling is one of the most frequent events in cancer (10.1097/PPO.0b013e31824d448c). In pancreatic cancer cell lines, PI3K/Akt pathway has been found to be implicated in chemotherapy drug resistance (10.1016/j.ctrv.2003.07.007). Mild hyperthermia involving temperature spiking to mimic fever has been shown by Choi et al, (Am J Pathol 172:367-777, 2008) to down-regulate the PI3K/Akt signalling pathway. Inhibition of PI3K/Akt by heat also has an inhibitory effect on neutrophil migration which down regulates the inflammatory response.

It is hereby hypothesised that the application of mild localised hyperthermia of a pulsed nature may inhibit PI3K helping the immune system in identifying the underlying tumour, promoting Langerhans cells and heat shock proteins to identify and present tumour fragments to the immune system causing a localised inflammatory response followed by eradication.

The emerging understanding is that hyperthermia treatments work at multiple levels via complex complementary mechanisms involving a variety of signaling and trafficking molecules.

WO2016123608 describes an imaging, guidance, planning and treatment system for treating cancer, the system exploiting Radio-Frequency Electrical Membrane Breakdown (“EMB” or “RFEMB”) to destroy the cellular membranes of unwanted or cancerous tissue, thereby ablating it and exposing tumour antigens and intra-cellular components which can have an immunologic effect on local or distant cancerous tissue.

US2017/0245929 provides methods, apparatuses and systems for non-invasive delivery of microwave therapy for the purpose of thermally ablating a target tissue.

US2014100266 refers to a method of treating cancer comprising using hyperthermia.

### SUMMARY

It is proposed here that microwave energy may be employed as a tri-action (heat shock promoting, immunostimulatory and tissue coagulation) treatment for melanoma skin lesions.

The microwave treatment mechanism may thermally instigate the production of heat shock factor HSF thus elevating the level of heat shock proteins in and proximal to diseased tissue as a means to invoke an immune response by associating the diseased tissue with the elevated heat shock proteins. This localised thermal increase can be achieved using a precise deposition of energy at the location of melanoma lesions which is readily achievable using microwave energy. This is applicable to any type, form or location of melanoma and the microwave energy can be delivered locally to precise locations of the body. This overcomes the systemic side effects often associated with chemotherapy, checkpoint inhibitors and vaccine based treatments.

As used herein, the term “melanoma” embraces any lesion with a melanoma aetiology. In particular, the term “melanoma” embraces all types and forms of “melanoma” including, for example, superficial spreading melanoma, nodular melanoma, lentigo maligna melanoma, acral lentiginous melanoma, amelanotic melanoma. Further, the term “melanoma” embraces any forms of the disease other than just “cutaneous melanoma”. For example, the term “melanoma” includes any type or form of the disease occurring in, for example, the eye, the vulva, the vagina, the rectum and/or the gastrointestinal tract.

It is also known that the incidence of melanoma is increased in immunosuppressed individuals—including, for example transplant patients. Within this population of patients, the incidence of HPV infection is also increased and this may impact on the occurrence of melanoma (see Le Mire et al. Melanomas in renal transplant recipients: Br J Dermatol. 2006 March; 154(3):472-7).

Localised microwave hyperthermia can be used to raise the temperature of the tissue containing the melanoma thus promoting a localised immune response specific to the cells in the volume.

The blood supply to the normal tissues of the body is maintained by an orderly and efficient vascular network. Blood vessels are regulated by the metabolic demand-driven balance of pro-angiogenic and anti-angiogenic molecular factors and a systematic network of lymphatic vessels which drain fluid and waste metabolic products from the interstitium. The overall effect allows adequate perfusion of oxygen and other nutrients to all cells.

In tumours, the aggressive growth of the neoplastic cell population and associated over expression of pro-angiogenic factors leads to the development of disorganised blood vessel networks that are fundamentally different from normal vasculature. Tumour vasculature is a complex labyrinth and typified by aberrant structural dynamics, vessels that are immature, tortuous, and hyperpermeable. As with the poorly developed vasculature, the accompanying lymphatic vessels are dilated, leaky and discontinuous leading to dilated fluid-engorged vessels. In combination, the ability of the tumour vasculature to deliver nutrients via blood vessels and remove waste products via the lymphatic system is drastically diminished. These aberrant micro-environmental conditions obstruct traditional therapeutic anti-cancer strategies that are delivered through the vasculature.

Microwave ablation can be used to coagulate or compromise the function of the blood supply to and within a melanoma tumour or lesion. The thermal insult and resultant disruption of blood supply to the diseased tissue can be used to destroy tissue supported by the feed network resulting in necrosis of the remaining tissue. Furthermore, the heating effects at hyperthermia levels can be used to enhance the efficacy of chemotherapy and radiotherapy in a range of tumours (as in Shevtsov M and Multhoff G (2016)).

Microwave energy is also suspected of having immunostimulatory properties and as such may also be used as a means to stimulate or enhance a host immune response. That immune response may be local to the area targeted by or exposed to the microwave energy. This phenomenon is discussed in more detail below but the stimulated immune response may be sufficient to facilitate the clearance and/or resolution of a melanoma.

Typically the dielectric properties of materials are measured relative to those of air and referred to as epsilon relative to air (Er) where air is Er=1. The dielectric properties have been researched by many authors (C. Gabriel, S. Gabriel and E. Corthout: The dielectric properties of biological tissues: I. Literature survey, Phys. Med. Biol. 41 (1996), 2231-2249) and it is known that the dielectric properties of diseased (for example, melanoma) tissue may differ when compared with normal tissue. The effect of electrical conductivity is the second factor that dictates the heating effects of microwaves in tissue. Thus, applicators that electrically match with the range of epsilon relative values and conductivity may be used to ensure energy is efficiently delivered into, for example, tissue infected by melanoma.

In biological tissues, the primary influence on conductivity is the water content which is known to vary from normal skin to for example malignant skin (DOI: 10.1109/IranianCEE.2014.6999804). With four times the water component, compared with healthy skin, tumours exhibit a conductivity of 2.94 S/m compared with 0.04 S/m for the healthy outer stratum corneum of skin. Additionally, the dielectric properties of melanoma skin may be Er 40-60 [same reference as above].

In view of the above, the dielectric properties/constant of diseased or malignant tissue (for example melanoma) may be higher than that of normal (not diseased or infected) tissue. As such, a tissue dielectric property/constant may form the basis of a procedure or method for the diagnosis or detection of a disease or condition, in particular a disease or condition which alters the dielectric properties of a tissue. For example, tissue dielectric properties may be exploited in methods of diagnosing or detecting diseases and/or conditions characterised by or causing, changes in levels of water in tissue(s). Thus, a dielectric property or constant may be exploited in a method of diagnosing or detecting a melanoma disease or associated malignant condition.

For example, the dielectric constant of test tissue may be compared to a control dielectric constant, wherein if the dielectric constant of test tissue is higher than that of the control dielectric constant, the test tissue may be malignant.

The test tissue may be any tissue (for example a skin or mucosal tissue) vulnerable to melanoma and/or suspected of developing melanoma. The test tissue may comprise tissue from a melanoma or other lesion suspected of having malignant aetiology. For example, the test tissue may be derived from a suspect mole or lesion.

A control dielectric constant may be that associated with a tissue which is not diseased and/or malignant (with melanoma). For example, where the tissue comprises melanoma, in accordance with the information presented above, a fixed value control dielectric constant may be in the range between Er 30 to Er 45.

A method of detection or diagnosis as described herein may be performed in vitro or in vivo. An in vitro method may be performed on a sample, for example a tissue biopsy or scraping.

In one application, a procedure or method for the diagnosis or detection of a disease or condition which alters the dielectric properties of a tissue (for example a malignant condition such as melanoma) may be conducted on a test tissue comprising a melanoma or other lesion suspected of having a malignant (melanoma) aetiology or a biopsy therefrom. Specifically, the dielectric properties of the tissue or sample may be compared to the corresponding control dielectric property from the same or a similar tissue, wherein if the dielectric property (constant) of the test tissue is higher than the corresponding control value, the test tissue may be diagnosed or identified as potentially comprising a malignancy such as melanoma.

A positive “melanoma” diagnosis may then lead to the use of any one of the microwave energy techniques or methods described herein to resolve or facilitate clearance of the melanoma (or a symptom thereof).

As a penetration depth of a few mm may be required, a microwave energy frequency in the range 2.45 GHz to 15 GHz is may be desirable as higher frequencies may not penetrate sufficiently. The power level and energy density of application will also affect the depth of penetration therefore lower frequencies may be used with appropriately designed applicators and treatment profiles. It may be advantageous to select a frequency that is not so low as to unnecessarily treat deeper tissues thus promoting invasive migration of an early melanoma.

The present invention is based on the finding that microwave energy may be used to treat melanoma and tumourous volumes.

Thus disclosed herein is a method of treating or preventing any type or form of melanoma, said method comprising administering to a subject, a therapeutically effective amount or dose of microwave energy.

The method may be applied or administered to a human or animal subject and to any tissue or region thereof. The subject may be any subject having or suffering from a form of melanoma or a subject predisposed or susceptible thereto.

The method may be applied or administered to the skin and/or mucosal tissues and/or to any ocular locations. Primary mucosal include the nasal cavity, sinuses, oropharynx, rectum, vulva (including clitoris and labia), vagina, oesophagus, stomach, small and large intestines, gallbladder, bile ducts, larynx, trachea, bronchi, lung, urethra, bladder, cervix, prostate, salivary glands, kidney, thyroid, thymus, pancreas, ovary, and adrenal glands.

Also disclosed herein is microwave energy for use in treating or preventing (or curtailing) melanoma.

One of skill will appreciate that in order to resolve, clear, treat or prevent a melanoma and/or to reduce the symptoms thereof, one or more treatments with microwave energy may be required.

Without wishing to be bound by theory, the inventors hypothesise that the induction of hyperthermia by targeted application of microwave energy, induces the production of heat shock proteins in diseased or damaged tissue. This may lead to the activation of antigen presenting cells (such as dendritic/Langerhans cells) which process antigen (including host, microbial or other foreign antigen) for presentation to T cells. Furthermore, the induction of hyperthermia may inhibit the PI3K pathway in Langerhans cells facilitating the induction of a potent host immune response. Localised thermal increase can be achieved using a precise deposition of energy to a diseased tissue (for example a lesion), readily achievable using microwave energy.

Further, it has been noted that when used at energy levels which do not result in any substantial tissue damage or ablation, subjects may exhibit good clinical responses (Ivan Bristow, Wen Chean Lim, Alvin Lee, Daniel Holbrook, Natalia Savelyeva, Peter Thomson, Christopher Webb, Marta Polak, Michael R. Ardern-Jones. Microwave therapy for cutaneous human papilloma virus infection. European Journal of Dermatology. 2017; 27(5):511-518. doi:10.1684/ejd.2017.3086). Again, without wishing to be bound by theory, the microwave energy based treatments described herein have been shown to induce an immune response which facilitates the (at least partial) clearance and/or resolution of a malignant disease and/or condition. The immune response may be a partially or fully protective immune response. The immune response may be a local response—that is a response which “local” to the site of microwave energy treatment or the disease and/or condition to be treated. The immune response may be a cell-based immune response involving the induction, proliferation and/or activation of one or more innate immune system mechanisms/pathways, immune system cells (T cells and the like) and/or cytokines. Thus, there is provided a method of stimulating an immune response in a subject, said method comprising administering a subject an amount or dose of microwave energy. Further, there is provided, microwave energy for use in a method of stimulating an immune response in a subject. As stated, the immune response may be a local response and/or a cell/cytokine based response. The stimulated immune response may itself be useful in the treatment and/or prevention of melanoma.

The subject may be any subject diagnosed as suffering from a melanoma. Additionally, or alternatively, the subject may be susceptible or predisposed to developing melanoma.

Thus, in one application, a method of raising an immune response via the use of microwave energy may be applied to a subject suspected of suffering from one or more melanoma lesions. In such circumstances, the method may involve applying microwave energy to a melanoma lesion so as to induce a local immune response. As stated, the raised immune response may then be sufficient to resolve or clear (at least partially) the one or more melanoma lesions.

The method of stimulating an immune response in a subject may exploit microwave energy applied at a level which does not result in any substantial macroscopic and/or histological changes in the tissue subjected to the microwave treatment. The microwave energy may be used at a level which is sub-apoptotic and/or which induces in human skin, mild macroscopic epidermal changes, microscopically minor architectural changes and slight elongation of keratinocytes without evidence of dermal collagen sclerosis. Higher levels of microwave energy may be used (for example 100J or >100 J) and this may result in more significant dermal and/or epidermal changes, including gross tissue contraction, spindled keratinocytes with linear nuclear architectural changes, sub-epidermal clefting and necrosis. The microwave energy may be applied at a sub-clinical level, that is an energy level which might not in itself result in resolution or clearance of the disease or condition (for example melanoma) affecting the targeted tissue.

The microwave energy may be applied at an energy of anywhere between about 1 J and about 500 J, for example, about 5 J to about 200 J. The microwave energy for use in a method of stimulating an immune response in a subject may be used at about 5 J, about 10 J, about 50 J, about 100 J or about 200 J.

The microwave energy for raising or stimulating an immune response may be applied for any suitable duration of time. The microwave energy may be applied for anywhere between about 0.1 s and about 1 minute. The microwave energy may be applied for about 1 s, about 5 s about 10 s, about 20 s or about 30 s. The microwave energy may be applied as multiple bursts or pulses of the same or different duration and/or of (or at) the same or different energy level. Each applied microwave energy burst/pulse may last for the same or a different duration. An applied amount of microwave energy may be described as a “microwave energy dose”

A subject may be delivered one or more microwave energy doses over a predetermined period of time. For example, a subject may be administered a single dose on 1 day or multiple doses over the same day, each dose being separated by a non-dosing period. Additionally or alternatively, a subject may be administered other doses on subsequent days. A treatment (comprising one or more microwave energy doses) may last a day, multiple days or one or more weeks months or years.

Where the melanoma lesion to be treated is small, for example, less than about 7 mm in diameter, a single dose may be applied to a single site within, on or to the lesion.

Where the lesion is larger, for example, larger than about 7 mm, the lesion may be applied multiple doses in a manner that ensures that the entire surface or area of the lesion has been exposed to microwave energy.

The immune response stimulated or induced by the methods described herein may comprise the activation of certain cell types, increased antigen presentation and/or modulated cytokine production. More specifically, an immune response stimulated or induced by a microwave energy based method of this invention may comprise keratinocyte activation, enhanced signalling between keratinocytes and dendritic cells which in turn enhances the level of antigen cross-presentation to CD8+T lymphocytes and enhanced IL-6 synthesis from keratinocytes.

One of skill will note that IL-6 is a pro-inflammatory cytokine which an important role in anti-viral immunity and which has been shown to induce rapid effector function in CD8+ cells. As such (and without wishing to be bound by theory), the microwave energy based methods described herein, which methods induce IL-6 synthesis, may facilitate the induction of a cytokine based anti-viral immunity.

Additionally, it has been shown that the microwave energy based methods might modulate certain other aspects of the host immune response. For example, the microwave energy may modulate interferon regulatory factor (IRF) expression. IRFs have been shown to be central to the regulation of an immune response. For example, IRF4 is essential for cytotoxic CD8+ T cell differentiation and its up-regulation in dendritic cells has been shown to enhance CD4+ differentiation, thereby potentially enhancing both CD8+ immunity and T cell help following microwave treatment.

Microwave energy may further modulate IL-10 production. Melanoma-derived IL-10 has been implicated in CD1 down-regulation and represents a possible mechanism through which melanoma cells can circumvent immune recognition. IL-10 is known to promote T cell exhaustion, and IL-10 blockade is known to reverse T cell dysfunction during chronic viral infections. Using microwave energy to modulate (for example decrease or inhibit) IL-10 production for a prolonged period may help the immune system re-establish control over melanoma development and spread.

Using microwave energy to modulate (for example decrease or inhibit) IL-4 for a prolonged period is also beneficial as both IL-4 and IL-10 are negative regulation factors in antitumour immune system response.

Thus the microwave energy based immunostimmulatory methods of this invention might be used to achieve any one or more of the following:
(i) modulation of immune cell activation;
(ii) modulation of antigen presentation;
(iii) modulation of cytokine expression;
(iv) modulation of IFR;
(v) modulation of ERK and PI3K pathway;
(vi) modulation of IL2;
(vii) modulation of IL4;
(viii) modulation of IL6;
(ix) modulation of IL-12;
(x) modulation of IL-17;
(xi) modulation of IL-10; and/or
(xii) modulation of T cells.

As stated, the stimulated immune response may be effective to facilitate the resolution and/or clearance of melanoma and lesions associated therewith.

One of skill will appreciate that unlike with vaccines and checkpoint inhibitors which are specific to a particular pathway, species, type or phenotype of malignant cells, microwave energy may be used to treat melanoma lesions and/or tumours/lesions irrespective of the number of the type or number of pathways involved and/or species, phenotype or type of melanoma. This is particularly important for a tumour which passes through phases of development where the proliferation mechanism changes thus their susceptibility to anti-tumour treatments can be hard to predict or match to available agents.

One of skill will appreciate that any method in which an immune response can be stimulated, enhanced or induced at the site of a disease and/or condition (for example melanoma), may represent an improvement over prior art methods which focus of the direct and individual treatment of each lesion. A method which stimulates, enhances or induces some form of local (cell and cytokine) based immune response at a single site of a melanoma may facilitate the clearance of a number of similar lesions in the same area.

Again, without wishing to be bound by any particular theory, the inventors suggest that the application of mild localised hyperthermia of a pulsed nature may inhibit PI3-K. This is important as PI3-K pathways are prominent in the pathogenesis of melanoma. For example, inhibition of PI3-K may help the immune system identify melanoma and promote Langerhans cells and heat shock proteins to identify and present melanoma-associated antigen to the immune system causing a localised inflammatory response. Thus, the immunostimulatory effect of microwave energy may facilitate resolution and/or clearance of melanoma.

Microwave energy according to this invention may have a frequency of between about 500 MHz and about 200 GHz. In other embodiments, the frequency of the microwave energy may range from between about 900 MHz and about 100 GHz. In particular, the frequency of the microwave energy may range from about 5 GHz to about 15 GHz and in a specific embodiment has a frequency of 8 GHz.

It should be understood that the methods of treatment described herein may require the use of a microwave energy having a single frequency or microwave energy across a range of frequencies.

The invention further provides an apparatus for use in treating melanoma, said apparatus comprising a microwave source for providing microwave energy and means for administering or delivering the microwave energy to a subject to be treated. The apparatus provided by this aspect of the invention may be used in any of the therapeutic methods described herein.

Advantageously, the microwave energy emitted or produced by the apparatus elevates or raises the temperature of the subject to be treated and/or stimulates a local immune response as described herein. In one embodiment, the microwave energy may cause targeted or localised hyperthermia in a tissue of the subject, including, for example, the skin and/or mucosal membrane. The temperature elevation may be localised to the surface of the skin and/or to the epidermal, dermal and/or sub-dermal layers thereof (including all minor layers that lie within).

The apparatus may further comprise means for controlling at least one property of the microwave energy produced by the microwave source. For example, the means may control or modulate the power, frequency, wavelength and/or amplitude of the microwave energy. The means for controlling the microwave energy may be integral with the apparatus or separately formed and connectable thereto.

In one embodiment, the microwave energy source may produce microwave energy at a single frequency and/or microwave energy across a range of frequencies. The means for controlling at least one property of the microwave energy may permit the user to select or set a particular microwave or microwaves to be produced by the apparatus and/or the properties of the microwave(s) produced.

The apparatus may further comprise means for monitoring the microwave energy produced or generated by the microwave source. For example, the apparatus may include a display indicating one or more properties of the microwave energy.

In one embodiment, the means for administering or delivering the microwave energy to a subject to be treated comprises an applicator formed, adapted and/or configured to deliver or administer microwave energy to the subject. The inventor has discovered that the dielectric properties of tissue affected by melanoma vary with respect to normal, healthy, tissue (i.e. tissue not affected by melanoma). As such, the means for delivering microwave energy may electrically match the range of epsilon relative values of the tissue affected by melanoma. In this way, it is possible to ensure efficient delivery of the microwave energy to the tissue.

The means for delivering the microwave energy to a subject may be connected to the microwave source via a flexible cable. In one embodiment the means for delivering the microwave energy to a subject (i.e. the applicator) may be connected to the microwave source via a flexible cable with locking connections having both microwave and signal data cables and may be reversible to enable connection to either port.

In one embodiment the invention provides an apparatus for delivering microwave energy to diseased tissue the apparatus comprising: —a microwave source for providing microwave energy, connectable to a system controller for controlling at least one property of the microwave radiation provided by the microwave source; and a monitoring system for monitoring the delivery of energy and an applicator means, for example an applicator device, for delivering microwave energy, wherein: —the applicator is configured to deliver precise amounts of microwave energy provided by the source at a single frequency or across a range of frequencies.

The applicator may not be an implant. That is to say, the microwave energy based treatments described herein may not be provided by means of an implanted microwave antennae.

A further embodiment of this invention provides a method for treating or preventing melanoma, said method comprising the administration or delivery of a therapeutically effective and/or immunostimulatory dose or amount of microwave energy to diseased tissue to produce, induce or elevate and immune response and/or levels of heat shock factor HSF to stimulate production of heat shock proteins in or near the tissue. In particular, the method for treatment may produce, induce or elevate levels of one or more heat shock proteins selected from the group consisting of HSP90, HSP72, HSP70, HSP65. HSP60, HSP27, HSP16 and any another heat shock protein(s) wherein: —the microwave energy promotes an association between the elevated heat shock proteins and the infected tissue so as to elicit an immune response against the infection. Additionally or alternatively, the microwave energy may be sufficient to induce a local immune response which comprises modulated cell and cytokine induction/expression/activation and enhanced antigen presentation.

The energy deposited into the target tissues are primarily expressed as heat. The therapeutic temperature range for hyperthermia (heat shock initiation) is lower than that of ablation where cell death is the desired outcome. The embodiments of both modes, hyperthermia and ablation are covered in this invention. The choice of energy and subsequent mode of biological response can be selected by the clinician and may reflect (1) the patient's co-treatment modes e.g. radiotherapy, chemotherapy, drug regime; (2) the classification of the melanoma; (3) the HPV presence; (4) if a biopsy has been confirmed or otherwise; (5) when dealing with second primary tumours and (6) the timing of the co-treatment modes.

In a further embodiment of the invention there is provided a method of treating, curtailing or preventing a disease and/or malignant condition (including any melanoma type) and/or a dermatological condition including those in which tissue (for example skin) is manifest with one or more malignant and/or tumourous region, a therapeutically effective and/or immunostimulatory dose or amount of microwave energy to infected or diseased tissue to facilitate resolution or clearance of the lesion and/or cauterises, coagulates, shrinks, blocks, ablates, damages, irritates, inflames or otherwise interferes with the normal operation of the capillaries supplying blood to the lesion and/or tumour. In one embodiment the lesion is a skin lesion including, for example, melanoma.

In another embodiment, the present invention provides a medical treatment regime comprising: —the application of microwave energy to diseased skin tissue (in other words skin comprising melanoma or skin exhibiting one or more symptoms associated with melanoma) to purposefully elevate levels of heat shock factor HSF to stimulate production of heat shock proteins in or near the tissue in particular HSP90, HSP72, HSP70, HSP65. HSP60, HSP27, HSP16 and any another heat shock protein(s) wherein the microwave energy promotes an association between the elevated heat shock proteins and the infected tissue with the intention to provoke an immune response against the melanoma

In a further embodiment, the present invention provides a medical treatment regime comprising: —the application of microwave energy into skin tissue to purposefully cauterise, coagulate, shrink, block, ablate, damage, irritate, inflame or otherwise interfere with the normal operation of the capillaries supplying blood thus causing necrosis of a melanoma lesion.

The disclosure further relates to the provision of treatments for melanoma comprising not only the microwave energy based treatments described herein, but also treatments comprising the use of:
(i) checkpoint inhibitors;
(ii) vaccines;
(ii) chemotherapy; and/or
(iii) radiotherapy.

For example, the disclosure may provide a combined treatment for melanoma type malignancies, the combined treatment comprising a microwave energy based treatment (according to a regimen or dose described herein) and, one or more other treatments selected from the group consisting of:
(i) checkpoint inhibitors;
(ii) vaccines;
(iii) chemotherapy; and/or
(iv) radiotherapy.

In all cases, the checkpoint inhibitor(s), the vaccine(s), the chemotherapy and/or radiotherapy used together or in combination with the microwave energy based treatments described herein, may be tailored, designed and/or formulated for the treatment and/or prevention of melanoma (as defined herein).

The phrase combination treatment should be taken to mean the administration of two or more distinct types of therapy—together. A combination treatment may comprise sequential use of the two or more treatments wherein one treatment is used before one or more of the others to be administered. A combination treatment may be taken to mean the use of two or more treatments together and at the same time.

Thus, the combination treatments described herein may take the form of methods in which microwave energy is used (i) concurrently with (that is “at the same time as”) or sequentially (that is before of after) treatment with one or more of:
(i) a checkpoint inhibitor;
(ii) a vaccine;
(iii) chemotherapy; and
(iv) radiotherapy.

One of skill will be familiar with the term “checkpoint protein” or “Immune checkpoint protein”; the term may be applied to the programmed death-1 (PD-1) protein or to the cytotoxic T lymphocyte antigen-4 (CTLA-4). Without wishing to be bound by theory, checkpoint proteins act to regulate the host (or self) immune system and act as an “off switch” to prevent T cells from attacking and damaging other “self” cells in the body. In the case of PD-1, the mechanism involves binding to PD-L1 a protein which is present on some cancer cells. Binding between PD-L1 on a tumour cell with PD-1 on a T-cell sends signals which prevent the immune system from attacking the tumour cells. Similarly, CTLA-4 also down-regulates immune responses by transmitting inhibitory signals to T cells.

Checkpoint inhibitors can be used to prevent these signals and to permit T cell to attack and destroy cancer cell. In other words, the use of a checkpoint inhibitor blocks any interaction between PD-L1 and PD-1 and/or the effects of CTLA-4; this inhibition prevents the cancer from evading the immune system.

Several checkpoint inhibitors have clinical utility, with many proving useful in the treatment or prevention of melanoma.

Antibodies (including monoclonal, or humanised antibodies) represent a particularly useful class of checkpoint inhibitor. Useful inhibitors include, for example:
Pembrolizumab (PD-1 inhibitor);
Nivolumab (PD-1 inhibitor);
Atezolizumab (PD-L1 inhibitor);
Avelumab (PD-L1 inhibitor);
Durvalumab (PD-L1 inhibitor); and
Ipilimumab (CTLA-4 inhibitor).

A checkpoint inhibitor (including any of the specific inhibitors described above) may be administered (or applied) with (for example together (concurrently with) or before or after) a microwave based treatment as described herein.

Thus, this disclosure provides a method of treating or preventing melanoma, said method comprising administering a subject in need thereof, microwave energy and a checkpoint inhibitor, wherein the microwave energy is administered:
(i) together (concurrently) with a checkpoint inhibitor;
(ii) before administration of a checkpoint inhibitor; or
(iii) after administration of a checkpoint inhibitor,

Without wishing to be bound by theory, it is suggested that microwave energy may act to up regulate signals and pathways within the adaptive arm of the immune system (for example, signals and pathways that lead to increased production of, for example, interleukin-12 (IL-12), tumour necrotise factor alpha (TNF-α) and other chemokines); under these conditions, the checkpoint inhibitors prevent the usual signals that might otherwise lead to a reduction in these cytokines/chemokines and the production stimulated by the use of microwave energy persists. Thus, the combination of microwave energy and checkpoint inhibitors is particularly advantageous as the microwave energy stimulates the production of anti-melanoma type cytokines/chemokines and the checkpoint inhibitors ensure that this stimulated production persists and production of these cytokines/chemokines is prolonged (as is exposure of a melanoma lesion to the same). Thus the combination of microwave energy and checkpoint inhibitors enhances the treatment of melanoma type cancers.

In contrast, and again without wishing to be bound by theory, microwave energy may depress, suppress or inhibit IL-10 or other anti-inflammatory cytokine production.

Melanoma is characterised by IL-10 expression and it is thought that the presence of this cytokine may inhibit the host anti-melanoma immune response. Additionally checkpoint inhibitor blockade of PD-1 on dendritic cells is known to increase production of IL-10 which correlates with a further increase in PD-1 expression which may account for the lack of durable response seen in the use of checkpoint inhibitors for some cancers.

As such, the combined use of microwave energy (to reduce IL-10 expression or reduce other anti-inflammatory cytokines within a melanoma) and the prior, concurrent or subsequent use of a checkpoint inhibitor to block anti-immune pathways and promote T cell-mediated elimination of target cells represents an improved treatment for melanoma and other cancers.

Vaccines for use in treating or preventing cancer are in development. A vaccine can be used to raise an immune response in a host. A vaccine for use in the treatment or prevention of cancer may be used to raise an immune response against one or more antigens expressed by a malignant cell. The resulting immune response can help the body restrain and kill malignant cells. In the case of melanoma, a vaccine may be used to generate antibodies specific to one or more of the antigens expressed on a melanoma cell.

Useful vaccines may be formulated to contain antigens expressed by a melanoma cell from a particular individual or patient. In this way the immune response is tailored to target an individual's cancer. Alternatively, a vaccine may comprise one or more antigens present within the general population of melanoma cells. An immune response rained by a vaccine of this type may be more general and may be used to generate anti-melanoma immune responses in a number of different people.

Accordingly, this disclosure provides a method of treating or preventing melanoma, said method comprising administering a subject in need thereof, microwave energy and a vaccine, wherein the microwave energy is administered:
(i) together (concurrently) with the vaccine;
(ii) before administration of the vaccine; or
(iii) after administration of the vaccine.

The term “vaccine” may embrace a melanoma vaccine. A melanoma vaccine may comprise one or more antigens from a melanoma cell. The vaccine may be administered once or a number of times as a “course”. For example, an immune response may develop over many months and require repeated. As such, when treating a melanoma using a vaccine, it may be necessary to administer a number of vaccine doses in order that the subject (the receiver of the vaccine (and microwave treatment)) reaches (or achieves) sufficient antibody (anti-melanoma antibody) titre. Each dose of vaccine may be provided as a combination treatment with a microwave energy based method described herein. With each vaccine dose, the microwave energy may be administered concurrently with the vaccine or sequentially as described above.

Chemotherapy remains a common therapeutic option. Chemotherapy is the general term applied to the use of drugs that kill cancer cells. Chemotherapy for the treatment of melanoma may comprise the use of one or more drugs selected from the group consisting of:
(i) Dacarbazine (also called DTIC)
(ii) Temozolomide
(iii) Nab-paclitaxel
(iv) Paclitaxel
(v) Cisplatin
(vi) Carboplatin
(vii) Vinblastine

Accordingly, this disclosure provides a method of treating or preventing melanoma, said method comprising administering a subject in need thereof, microwave energy and a chemotherapeutic agent, wherein the microwave energy is administered:
(i) together (concurrently) with the chemotherapeutic agent;
(ii) before administration of the chemotherapeutic agent; or
(iii) after administration of the chemotherapeutic agent.

Treatment of melanoma by chemotherapy may require one or more doses of the chemotherapeutic agent. At each dose, the chemotherapeutic agent may be administered in combination (sequentially or concurrently) with a microwave energy based treatment as described herein.

Radiation therapy may also be used in the treatment and/or prevention of melanoma (in particular desmoplastic melanoma). Radiotherapy may be used after surgery—as a follow on or additional treatment. Radiotherapy most often takes the form of “external beam radiation therapy”, which focuses radiation from a source outside of the body on the melanoma. Stereotactic radiosurgery (SRS) may be used treat tumours that have spread to the brain. In this technique, high doses of radiation are aimed at the tumour(s) in one or more treatment sessions. There are 2 main ways to deliver SRS:
(i) a Gamma Knife® machine focuses about 200 beams of radiation on the tumour from different angles over a few minutes to hours. The head is kept in the same position by placing it in a rigid frame; or
(ii) a linear accelerator (a computer controlled machine that creates radiation) moves around the head to deliver radiation to the tumour from many different angles over a few minutes. The head is kept in place with a head frame or a plastic face mask.

Stereotactic body radiation therapy (SBRT) is a further variation which also uses a linear accelerator but which is used to treat tumours in other parts of the body (i.e. not in the brain).

Accordingly, this disclosure provides a method of treating or preventing melanoma, said method comprising administering a subject in need thereof, microwave energy and radiotherapy, wherein the microwave energy is administered:
(i) together (concurrently) with the radiotherapy;
(ii) before administration of the radiotherapy; or
(iii) after administration of the radiotherapy.

It should be noted that treatments based upon the use of checkpoint inhibitors, vaccines, chemotherapy and radiotherapy may be used after, for example, surgery to remove a tumour (and/or to biopsy the same).

Microwave energy-based treatments (as described herein) may be regarded as “adjuvant” treatments, that is treatments used to supplement, enhance or improve existing treatments. Thus, the disclosure provides microwave energy for use, or the use of microwave energy, as an adjuvant treatment for:
(i) checkpoint inhibitor
(ii) vaccine
(iii) chemotherapy; and/or
(iv) radiotherapy

based treatments.

The term “adjuvant” may be taken to mean that the microwave energy based method of this disclosure modify or alter the effect of one or more other treatments administered in combination therewith. For example, by combining any of a checkpoint inhibitor based treatment, a vaccine based treatment, a chemotherapeutic agent and/or radiotherapy with the microwave energy based treatments described herein, it may be possible to modify or alter (for example improve, enhance or prolong) the effects of the checkpoint inhibitor based treatment, vaccine based treatment, a chemotherapeutic agent and/or radiotherapy. By administering checkpoint inhibitors, vaccines, chemotherapeutic agents and/or radiotherapy in combination with microwave energy based treatments as described herein) it may be possible to reduce the total amount of checkpoint inhibitor, vaccine, chemotherapeutic agent and/or radiotherapy required to achieve a suitably therapeutic outcome.

In one embodiment, the combination therapy described herein does not exploit a Dbait molecule (in other words a nucleic acid molecule mimicking a double strand break).

Further, the combination therapies described herein may not use a combination of a microwave energy based treatment as described herein and a heat-killed Mycobacterium (for example Mycobacterium obuense) as an immunomodulatory compound.

The microwave treatments described herein may be used to promote prolonged down-regulation of IL-10, IL-4 and/or other anti-inflammatory cytokines, and/or other negative regulation factors in the anti-tumour (melanoma) immune system response, to enhance the durability of action or use of checkpoint inhibitors with or before and/or after the microwave based treatment.

### BRIEF DESCRIPTION OF THE DRAWINGS

Embodiments of the invention are now described, by way of non-limiting example, and are illustrated in the following figures, in which:—

FIG. 1 is a schematic illustration of an embodiment of a microwave treatment system.

FIG. 2(a) is a schematic illustration of a Papilloma caused by HPV infection.

FIG. 2(b) is a schematic illustration of the terminal differentiation pathway of epidermal cells infected by the HPV virus.

FIG. 3 is a schematic illustration of functional representation of a microwave treatment system for application to treat Papilloma or other dermatological lesions.

FIG. 4 is a schematic illustration of a microwave treatment system applicator according to an alternative embodiment.

FIG. 5 is a schematic illustration of the microwave activation of the heat shock response.

FIG. 6(a) is a schematic illustration of measurement results of Er vs. Frequency for verrucae tissue for a sample population.

FIG. 6(b) is a schematic illustration of results of Loss tangent vs. Frequency for verrucae tissue for a sample population.

FIG. 7 is a schematic illustration of Er vs. Frequency for various plantar tissues for a sample population.

FIG. 8 is a schematic illustration of the statistical analysis of the sample median of verrucae tissue for a sample population.

FIG. 9 is a schematic illustration of a comparison of the statistical analysis of sample medians of various plantar tissues for a sample population.

FIG. 10(A) shows a clinical image of plantar wart pre-microwave treatment (left), after one treatment (middle) and after two treatments (right). FIG. 10(B) shows a clinical image of plantar wart pre-microwave treatment (left), after one treatment (right). FIG. 10(C) is a graphical representation of data from an Intention to treat analysis, in which 32 patients with 54 HPV foot warts were treated by microwave therapy over 5 visits: baseline, 1 week, 1 month, 3 months, and 12 months. Resolved warts were enumerated.

FIG. 11(A) shows histological analysis of human skin treated with microwave visualised at the epidermis/papillary dermis (top and bottom panels), or deep dermis (middle panels). Skin was subject to microwave therapy (0-200 J), before punch excision. Tissue was cultured for 1 hour before fixation and paraffin embedding. H&E or TUNEL staining. Original magnifications, ×20. FIG. 11(B) shows histological analysis of human skin treated with microwave visualised at the epidermis/papillary dermis (upper panels), or deep dermis (lower panels). Skin was subject to various liquid nitrogen therapy for 5, 10, or 30 seconds, before punch excision. Tissue was cultured for 1 hour before fixation and paraffin embedding. H&E or TUNEL staining. Original magnifications, ×20. FIG. 11(C) is a graphical representation of data obtained following microwave therapy (top panel) or cryotherapy (bottom panel), skin samples (in triplicate) were excised and cultured in media for 1 or 16 hours before harvesting supernatant for measurement of lactate dehydrogenase (LDH) by ELISA. FIG. 11(D) shows images in which skin was subject to microwave therapy (150 J), before punch excision at the margin of the treated zone. Tissue was cultured for 1 hour before fixation and paraffin embedding. H&E stain. Original magnifications, ×10. FIG. 11(E) shows images in which skin was subject to microwave therapy (150 J), before punch excision. Tissue was cultured for 1 hour before fixation and paraffin embedding. H&E stain showing deep dermis. Original magnifications, ×100. Representative of 3 independent experiments.

FIG. 12(A), Left panel shows flow cytometric analysis of viable keratinocytes (% of total cells) indicated by negative staining with the amine reactive viability dye LIVE/DEAD after control, microwave (5-150 J), or LPS/IFNg treatment. Keratinocytes were treated, then kept in culture for 24, 48 or 72 hours before analysis. FIG. 12(A), Right panel Shows flow cytometric analysis of keratinocyte viability after microwave therapy or control depicted as a histogram. X-axis: MFI LIVE/DEAD stain; y-axis: cell count. FIG. 12(B) shows flow cytometric analysis of HLA-DR, ICAM-1, CD40 or CD80 expression on viable keratinocytes. Keratinocytes were treated with microwave therapy (5-150 J), LPS/IFNg, or nil (control), rested in culture for 24, 48 or 72 hours, before analysis of the viable population. FIG. 12(C) shows flow cytometric analysis of CD86, CD80, and CD40 expression on viable monocyte derived dendritic cells (moDCs). Keratinocytes were treated with microwave therapy (5-150 J), LPS/IFNg, or nil (control), rested in culture for 8 hours then washed. They were left in culture for the remaining time until 24 or 48 hours, before transfer of supernatant onto moDCs. MoDCs were incubated for 24 hours before harvesting for analysis. Data representative of 3 independent experiments. Mean+SD.

FIG. 13(A) shows ELISpot assay of IFN-γ production by HPV-specific CD8+ cells following co-culture with HPV peptide pulsed moDCs primed by supernatant from untreated, microwave treated (150 J) or cryotherapy (cryo) treated keratinocytes. moDCs were primed with supernatant for 24 hours prior to pulsing with control or HPV peptide for 2 hours. Pulsed moDCs were then cocultured with an HLA-matched CD8+ HPV-specific T cell line before assay with IFN-γ ELISpot. Statistical significance determined using the Holm-Sidak method, with alpha=5%. Data representative of 3 independent experiments. Mean+SD. FIG. 13(B) shows ELISpot assay of IFN-γ production by HPV-specific CD8+ cells following co-culture with HPV E16 protein pulsed moDCs primed by supernatant from untreated, microwave treated (150 J) or cryotherapy (cryo) treated keratinocytes. moDCs were primed with supernatant for 24 hours prior to pulsing with control or HPV peptide for 2 hours. Pulsed moDCs were then co-cultured with an HLA-matched CD8+ HPV-specific T cell line before assay with IFN-γ ELISpot. Statistical significance determined using the Holm-Sidak method, with alpha=5%. Data representative of 3 independent experiments. Mean+SD. FIG. 13(C) shows flow cytometric analysis of intracellular HSP-70 expression on viable keratinocytes after microwave therapy or control depicted as a histogram. Primary human keratinocytes were treated with microwave therapy (150 J), or nil (untreated), rested in culture for 24 hours, before analysis. X-axis: MFI anti-HSP-70; y-axis: cell count. FIG. 13(D) shows ELISA of IL-6, TNFα, IL-1β production by primary human keratinocytes 24 h after treatment with microwave therapy (150 J), LPS/IFNg, cryotherapy or nil (untreated). FIG. 13(E) shows expression changes of IRF1 and IRF4 in human skin with microwave therapy (25 J and 150 J) by qPCR.

### DETAILED DESCRIPTION OF EMBODIMENTS

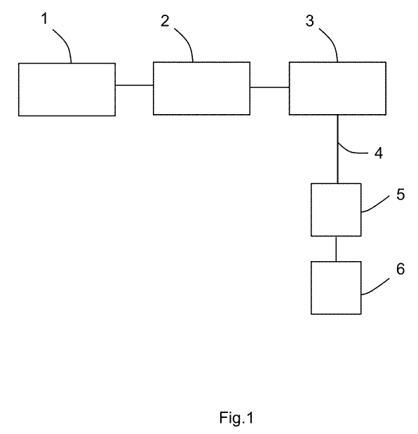

An embodiment of a microwave power generator system for medical applications is illustrated in FIG. 1 The apparatus comprising: —a microwave source for providing microwave energy 1, connectable to a system controller 2 for controlling at least one property of the microwave radiation provided by the microwave source; and a monitoring system 3 for monitoring the delivery of energy and an interconnecting cable 4 and an applicator hand piece 5 and a removable applicator means 6, for example an applicator device, for delivering microwave energy, wherein: —the applicator is configured to deliver precise amounts of microwave energy provided by the source at a single frequency or across a range of frequencies.

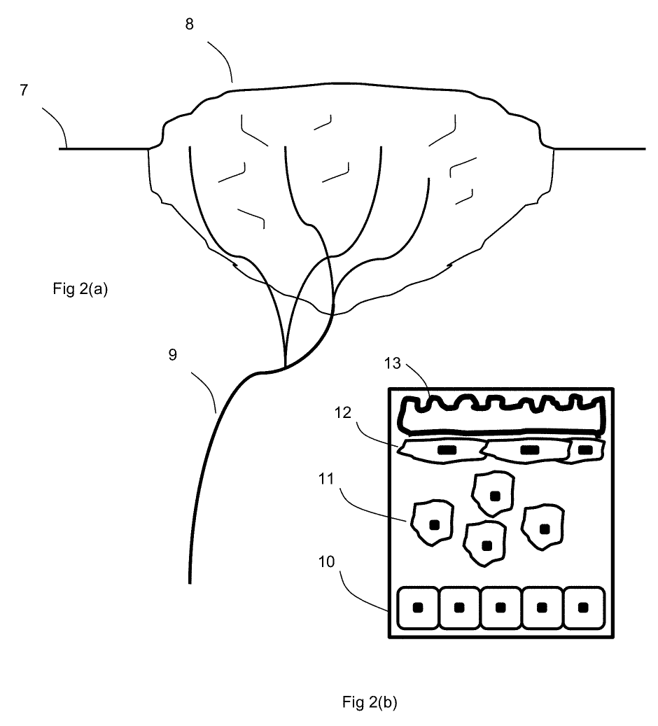

A typical HPV infection is illustrated in FIG. 2(a), this comprises the normal tissue 7, the papilloma surface 8, the capillary feed network 9. The terminal differentiation pathway of epidermal cells infected by the HPV virus is illustrated in FIG. 2(b) basal cells 10 become infected with the HPV virus leading to viral replication in the stratum spinosum 11 followed by assembly of virus particles in the stratum granulosum and release of virus particles in the in the stratum corneum (papilloma surface).

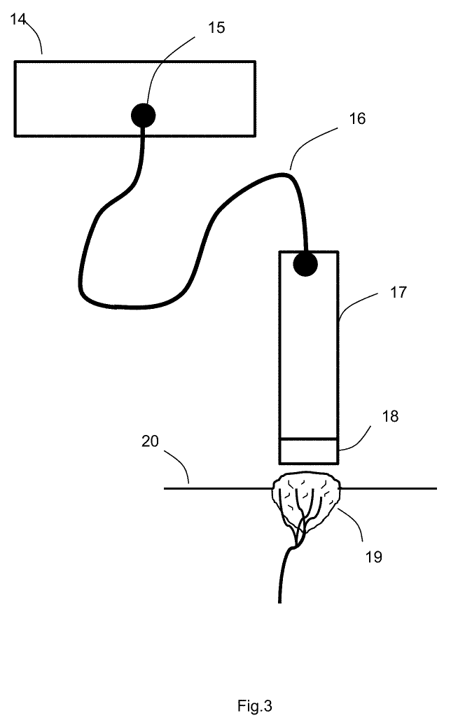

FIG. 3 shows the components of an apparatus according to an embodiment of the present invention, the components shown separately for ease of reference. The apparatus comprises a generator system 14 with a locking microwave connection 15 to a flexible microwave cable 16 connected to a hand piece 17 (which may have the same type of locking connection) which accepts an applicator component 19. The applicator component is designed to match to the tissue properties of the papilloma 20 and not match to the normal tissue 18. The cable 16 may include both microwave and signal data cables and may be reversible to enable connection to either port.

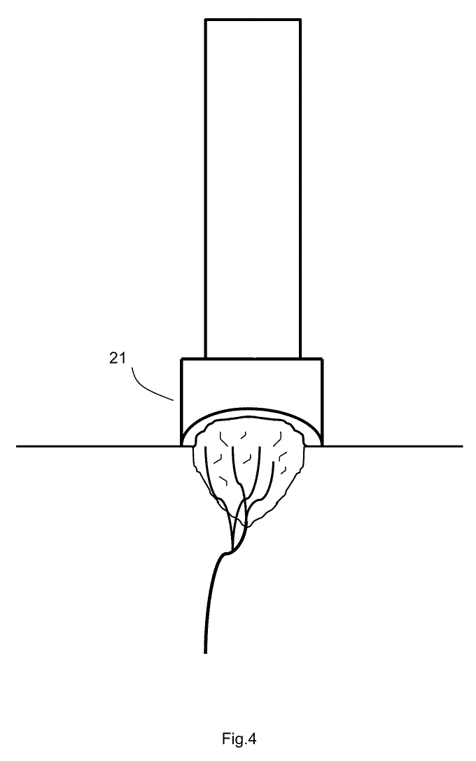

An alternative embodiment of an applicator 21 is illustrated in FIG. 4 with the component having a domed or enclosing surface compatible with raised or curved lesions.

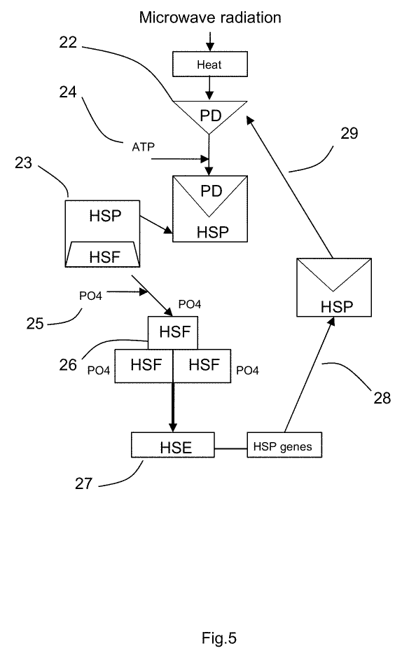

The method of inducing a microwave heat shock responses is illustrated in FIG. 5. In this illustration microwave radiation creates a thermal stress which results in protein denaturation 22. Heat shock proteins (HSP) are normally bound to heat shock factors (HSF) (23), but dissociate in the presence of denatured proteins (PD). Once dissociated, HSPs bind to the denatured proteins by rapid release. This requires Adenosine Triphosphate (ATP) 24. Further HSPs are generated when HSFs phosphorylate (PO4) (25) and trimerize (26). These trimers bind to heat shock elements (HSE) 27 that are contained within the promoters of the HSPs and generate more protein 28. Newly generated HSPs can then free to bind more denatured proteins 29.

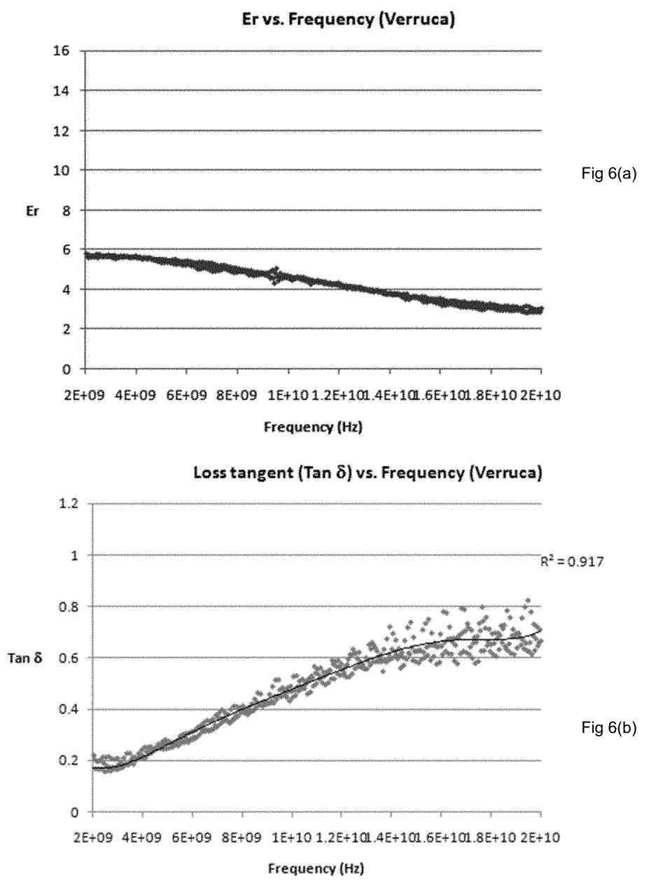

The measured dielectric properties for the sample population median versus frequency of plantar verrucae are reported in FIGS. 6(a) and 6(b). Measured results for ER and loss tangent versus frequency from 2 GHz to 20 GHz are reported. The measurements were made using an Agilent PNA-L Network Analyzer connected to an Agilent 85070E dielectric measurement system with the 85070E Performance Probe Kit (Option 050) measuring from 300 kHz to 20 GHz. Deionised water and air were used as dielectric references for calibration.

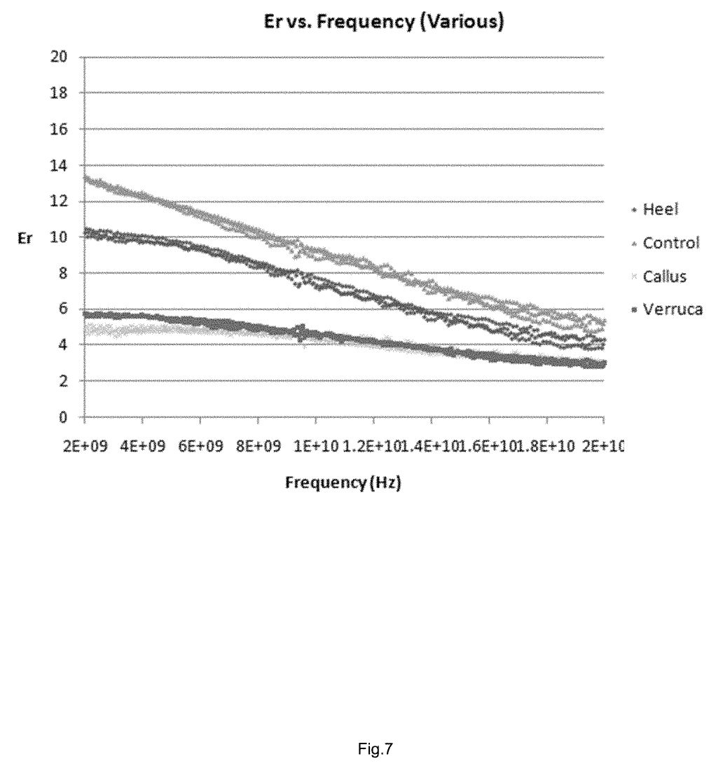

The measured dielectric properties for the sample population (median taken across the population) versus frequency for various plantar tissues are reported in FIG. 7. Measured results for Er versus frequency from 2 GHz to 20 GHz are reported illustrating demarcation between each tissue type.

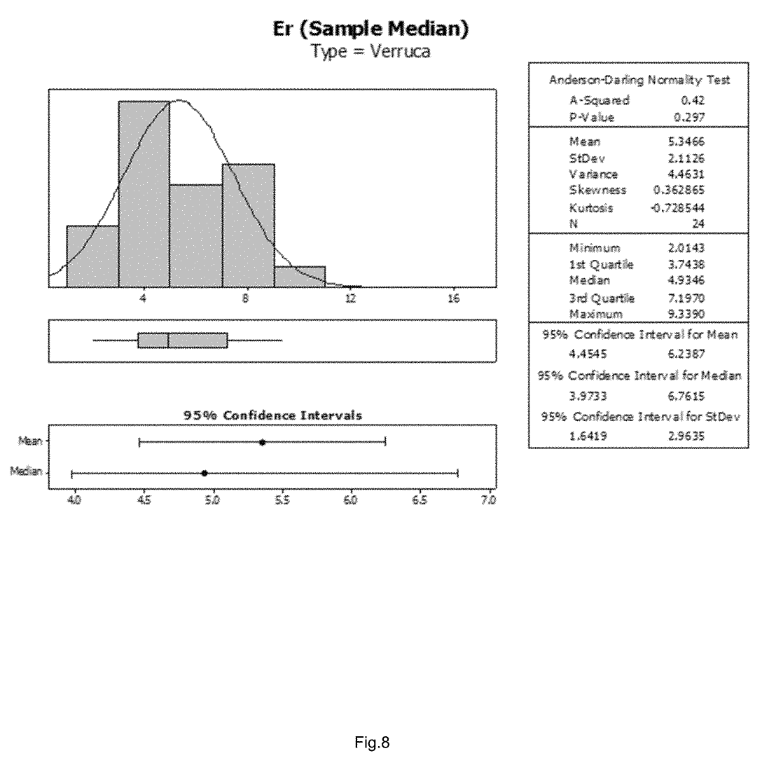

Statistically analysed dielectric property values taken over a sample population (using the median of the measurement range 7.5-8.5 GHz taken from each sample) for Verrucae tissue is presented in FIG. 8. The median Er value was measured at 4.93 for this dataset.

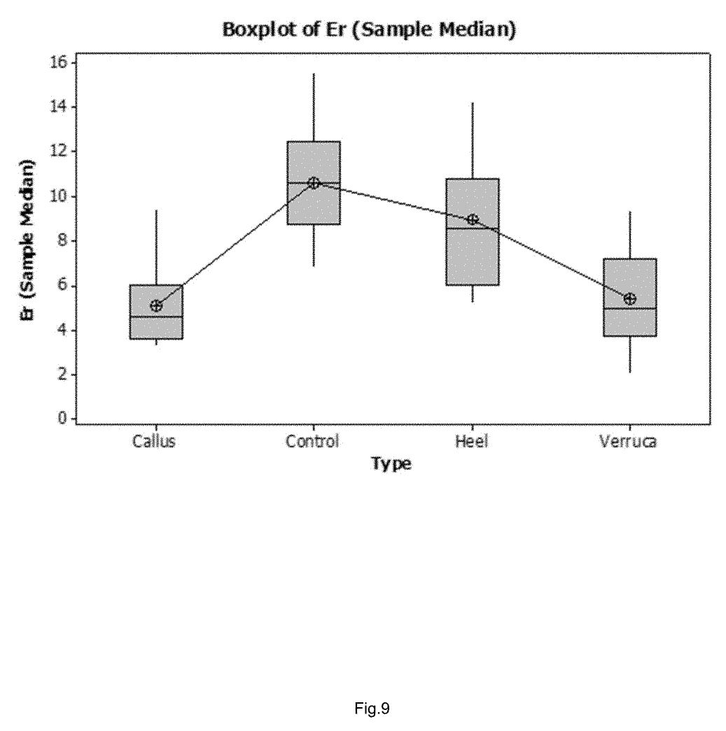

With reference to FIG. 9 a comparison of statistically analysed measurements of the dielectric properties of various plantar tissues over a sample population (using the median of the measurement range 7.5-8.5 GHz taken from each sample) is illustrated.

Microwave Therapy for Cutaneous Human Papilloma Virus Infection

Methods and Materials

Patients and In Vivo Microwave Treatment

Patients with treatment-refractory plantar warts were excluded if they had a pacemaker fitted, were pregnant or breast feeding, had any metal implants within the foot or ankle, suffered any known disease or condition affecting their immune function or their capacity to heal. Adverse events were categorised as being specifically associated with the microwave procedure, or unrelated. A complete examination of the affected area was undertaken at each study visit. At the conclusion of the treatment session all patients were given an advice information sheet advised to report any complications. No post-operative dressing was required and patients were advised to subsequently undertake normal everyday activities as usual with no restrictions.

A total of 32 patients with 54 foot warts were enrolled into the study. Of the 32, 17 were males and 15 females. Ages ranged from 22-71 years with a mean age of 44.79 years (sd 13.019]. Of the 54 lesions, 16 were reported as single lesions, and 38 as multiple type lesions (including mosaic verrucae). The average lesion duration was 63 months (5.25 years) with a range of 2-252 months (<1-21 years). The mean lesion diameter was 7.43 mm (sd 6.021), ranging from just 2 mm to 38 mm in diameter.

The procedure was performed in an out-patient setting, with standard podiatric facilities. The Swift device settings were titrated up as tolerated to 50 J over a 7 mm² application area (7.14j/mm²). The microwave energy was delivered to the affected area over 5 s duration (50 J delivered as 10 watts for 5 s). Lesions which were <7 mm in diameter were treated with one application of the probe at a single treatment session whilst lesions >7 mm were underwent multiple applications until the entire surface of the wart had been treated.

Clinical assessments were performed at baseline and at 1 week, 1 month, 3 months, and 12 months after treatment by a podiatrist experienced in the management of plantar warts. Response to treatment was assessed by the same investigator as ‘completely resolved’ or ‘unresolved’. Complete resolution was indicated by fulfilling three criteria: i. lesion no longer visible, ii. return of dermatoglyphics to the affected area, iii. no pain on lateral compression. Pain was assessed using a 10 point visual analogue scale.

Human Skin and Ex Vivo Microwave Treatment

Normal skin samples were acquired from healthy individuals after obtaining informed written consent with approval by the Southampton and South West Hampshire Research Ethics Committee in adherence to Helsinki Guidelines. Skin samples were treated immediately ex-vivo with microwave (Swift s800; Emblation Ltd., UK) or liquid nitrogen therapy and treated skin excised. Excised skin was sent for histological analysis or placed in culture media.

Histological analysis with hematoxylin and eosin (H&E) tissue sections were undertaken following fixation and embedded in paraffin wax. DNA damage was assessed by staining for single stranded and double stranded DNA breaks by TUNEL assay using the ApopTag® In Situ Apoptosis Detection Kit (Millipore, UK). Following culture, supernatants were collected and analysed for lactate dehydrogenase release using the Cytotoxicity Detection Kit (Roche applied science) as a measure of apoptosis.

Culture and In Vitro Microwave Treatment.

Human skin and HaCaT keratinocytes were cultured in calcium-free DMEM (ThermoFisher Scientific) with 100 U/mL penicillin, 100 μg/mL streptomycin, 1 mM sodium pyruvate, 10% fetal bovine serum (FBS) and supplemented with calcium chloride at 70 μM final concentration.

Lymphocytes were cultured in RPMI-1640 media with 100 U/mL penicillin, 100 μg/mL streptomycin, 1 mM sodium pyruvate, 292 μg/mL L-glutamine, supplemented with 10% FBS or 10% heat inactivated human serum (HS). HaCaT cells were cultured at sub-confluency to avoid cell differentiation and used in assays at passage 60-70. Cells were plated at 2.5×103 cells/well in 96-well flat plate (Corning Costar) and cultured overnight to reach confluence. HaCaTs were washed once with PBS before treatment with 150 J microwave, liquid nitrogen (10 s), heat (42° C. preheated media) or with LPS+ IFN-γ (1 ng/mL+1000 U/mL). Cells were cultured for 24 h before supernatants were harvested.

For HPV-specific T cell lines, PBMCs were isolated from HLA-A2 individuals as previously described 11. PBMCs were seeded at 2-4×106 cells/well in 24-well culture plate and 10 μg/mL of 9mer HLA-A2 restricted HPV16 epitope LLM (LLMGTLGIV) 12 was added, cells were cultured in 1 mL RPMI+10% HS. On day 3, cells were fed with RPMI+10% HS+IL-2 (200 IU/mL), and then fed again on day 7 or when needed. After day 10, HPV-specific T cells were harvested for cryopreservation before testing against HPV in ELISpot assays.

Monocyte derived dendritic cells (moDCs), CD14+ cells were positively isolated from PBMCs by magnetic separation using CD14 microbeads (Milentyi Biotec), according to manufacturer's protocol. Cells were washed and resuspended in RPMI+10% FBS+250 U/mL IL-4 and 500 U/mL GM-CSF. At day 3, cells were fed with RPMI+10% FBS+IL-4 and GM-CSF, and then harvested on day 5 for use in functional assays.

In vitro, microwave therapy of cell cultures was delivered through the base of the plastic culture dish and showed a linear dose response between the energy delivered and thermal induction (not shown). Utilising the equation E=m×c×θ (E=energy transferred, J; m=mass, kg; c=specific heat capacity, J/kg ° C.; θ=temperature change, ° C.), we calculated that in our system the 150 J Swift programme delivered 15.58 J (s.d. 0.921) through the plastic to the culture.

ELISpot, Flow Cytometry and qPCR

Keratinocytes were treated with microwave at various energy settings before removal of supernatant at various time points. MoDCs were treated overnight with keratinocyte supernatant, then washed twice before incubation with 10 μg/mL LLM peptide for 2 hours before a further wash.

Human IFN-γ ELISpot (Mabtech, Sweden) was undertaken as per manufacturer's protocol and as reported previously 11. 1×103 moDCs were plated with autologous HPV peptide-specific T cells at 1:25 ratio. Spot forming units (sfu) were enumerated with ELISpot 3.5 reader (AID, Germany). MoDCs were treated with HaCaT supernatant and harvested at 24 hours for flow cytometric analysis of cell phenotype. Cells were stained with violet LIVE/DEAD stain (Invitrogen) for 30 min at 4° C., then washed with PBS+1% BSA and stained with antibodies PerCP-Cy5.5 anti-HLA-DR, FITC anti-CD80, FITC anti-CD86, PE anti-CD40, all purchased from BD, for 45 min at 4° C. Cells were washed then resuspended in PBS+1% BSA and analysed using the BD FACSAria and the FlowJo v10.0.08 analysis software.

The expression of chosen genes was validated with quantitative PCR, using the TaqMan gene expression assays for target genes: YWHAZ (HS03044281_g1), IRF1 (Hs00971960_m1), IRF4 (Hs00543439_CE) (Applied Biosystems, Life Technologies, Paisley, UK) in human skin treated as indicated. RNA extraction (RNeasy micro kit, Qiagen) and reverse transcription (NanoScript kit; Primer Design, Southampton, UK) were carried out accordingly to the manufacturer's protocol.

Results:

Treatment of Human Papilloma Virus Infection in Humans with Microwave Therapy

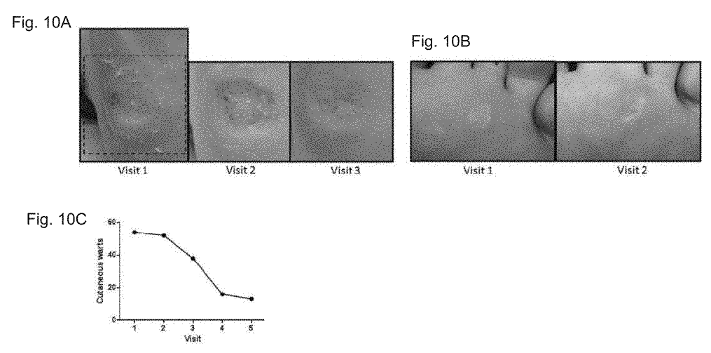

From January 2015 to September 2015 at the University of Southampton, we enrolled 32 patients with severe, treatment-refractory plantar warts. The diagnosis of plantar wart was confirmed by a podiatrist experienced in management of such lesions. A clinically significant wart was defined as >1 year duration, which had failed at least two previous treatments (salicylic acid, laser, cryotherapy, needling and surgical excision). In each patient, the most prominent plantar wart (most severely affected) was targeted for treatment (FIG. 10 a, b).

At the end of the study period, of the 54 warts treated, 41 had resolved (75.9%), 9 remained unresolved (16.7%), 3 warts (n=2 patients) had withdrawn from the study (5.6%) and 1 patient (with 1 wart) was lost to follow up (1.9%). The mean number of days to resolution 79.49 days (sd 34.561; 15-151 days). 94% of resolving lesions had cleared after 3 treatments (FIG. 10c). No significant difference in resolution rates were observed between males and females (p=0.693) was observed.

Microwave Treatment of Human Skin

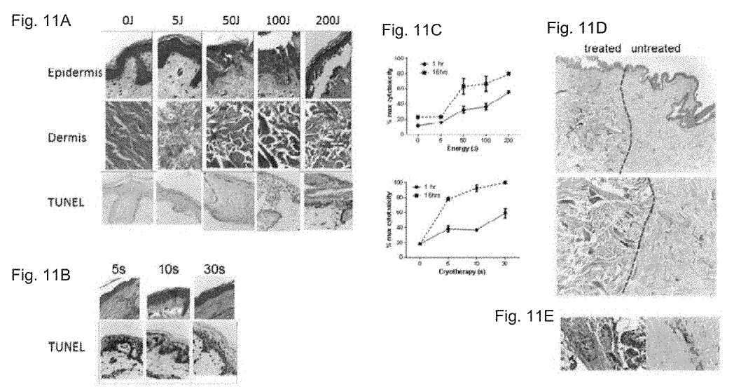

Human skin has not been previously treated with microwave therapy, therefore, we proceeded to undertake a full histological analysis. Skin removed during routine surgery was sectioned 1 hour after treatment ex vivo. Neither macroscopic, nor histological changes were noted with the lowest energy setting (5 J). At 50 J, mild macroscopic epidermal changes only were noted, and microscopically minor architectural changes, and slight elongation of keratinocytes were seen without evidence of dermal collagen sclerosis. At higher energies (100, 200 J) gross tissue contraction was visible macroscopically. Microscopic changes in the epidermis were prominent, showing spindled keratinocytes with linear nuclear architectural changes and subepidermal clefting (FIG. 11a).

Dermal changes were prominent at energies of 100 J and above and showed a homogenous zone of papillary dermal collagen, thickened collagenous substances, accentuation of basophilic tinctorial staining of the dermal collagen with necrotic features (FIG. 11a). These features are similar to electrocautery artefacts and suggest the potential to induce scarring at >100 J. Histological analysis both at 16 h and 45 h showed similar changes (not shown).

In clinical practice, cryotherapy is delivered to the skin by cryospray, which is time-regulated by the operator. In contrast to microwave therapy, minimal epidermal or dermal architectural change was identified with cryotherapy at standard treatment duration times (5-30 s), but did show a dose dependent clumping of red blood cells in vessels (FIG. 11b).

Tissue release of LDH acts as a biomarker for cellular cytotoxicity and cytolysis. To examine the extent of cell death induced by microwave irradiation, human skin was treated with 0, 50, 100 or 200 J before punch excision of the treated area and incubation in medium for 1 hour or 16 hours. Measurement of LDH revealed a dose dependent induction of tissue cytotoxicity with increasing microwave energies (FIG. 11c). In line with the lack of histological evidence of cellular damage, at 5 J, cytotoxicity of microwave application was equivalent to control. Early cytotoxicity was not prominent at 50 J, but became more evident after 16 hours. Higher energy levels induced more prominent cytotoxic damage. In contrast to microwave therapy, liquid nitrogen treatment of skin induced cytotoxicity at the lowest dose both at 1 hour and 16 hours.

Terminal deoxynucleotidyl transferase dUTP nick end labelling (TUNEL) identifies cells in the late stage of apoptosis. Analysis at 0, 5, 50, 100 and 200 J identified increased cellular apoptosis in the epidermis above 100 J (FIG. 11a). In contrast, cryotherapy with a liquid nitrogen spray applicator directly to the skin as used in clinical practice, even at a very short treatment time (5 s) induced significant epidermal and dermal DNA fragmentation (FIG. 11b).

The physics of microwave therapy suggests a tight boundary between treated and untreated tissue with minimal spreading of the treated field. This was borne out histologically by a clear demarcation between treated areas extending vertically from the epidermis through the dermis (FIG. 11d). Examination of the dermis showed that microwave therapy modified skin adnexae inducing linear nuclear architectural changes in glandular apparatus, microthrombi, fragmented fibroblasts and endothelial cells (FIG. 11e).

Microwave Induction of Immune Responses in Skin

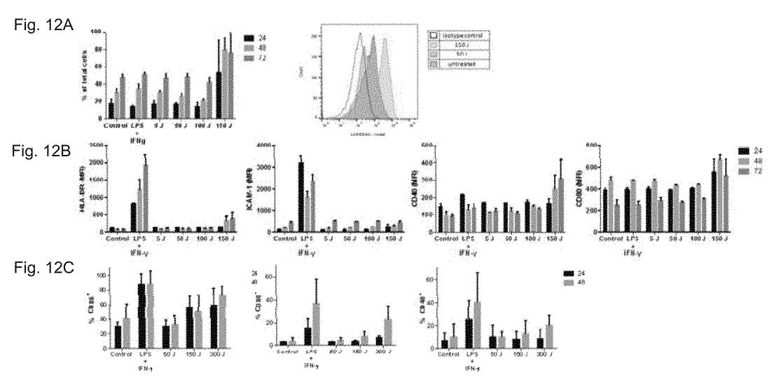

We first examined the response of keratinocytes to microwave therapy in vitro. Keratinocyte apoptosis was induced by microwave therapies above 100 J in vitro (FIG. 12a). Only above the apoptotic threshold were surface phenotypic changes of cellular activation noted in viable cells with increased expression of HLA-DR, CD40 and CD80 (FIG. 12b). Next, we utilised a model of skin cross talk of keratinocyte signalling to dermal dendritic cells. Keratinocytes were treated with microwave therapy as above, and washed after 8 hours to remove dead or apoptotic cells. Treated keratinocytes were then incubated for a further 16 hours before supernatant collection to prime monocyte derived dendritic cells (moDCs) which had not been directly exposed to microwave therapy. This showed a potent induction of moDC activation with increased expression of CD86, CD80 and to a lesser extent CD40 (FIG. 12c).

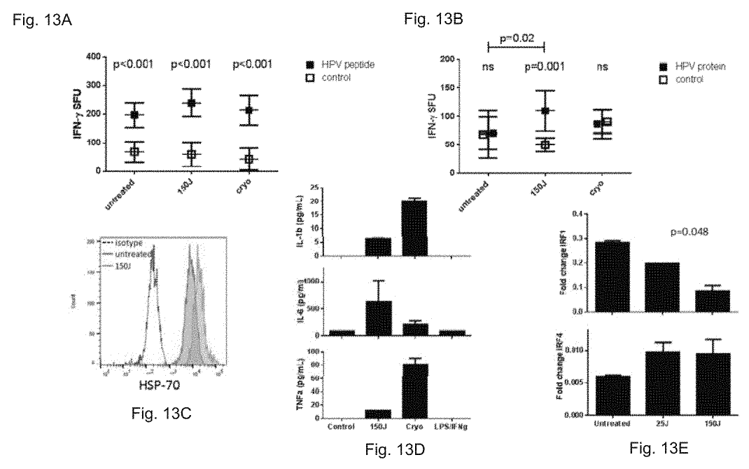

We next set out to test the functional outcome on skin dendritic cells following microwave treatment of keratinocytes. Keratinocytes were untreated, microwave, or cryotherapy treated before supernatant harvesting. Supernatant primed DCs were pulsed with a 9 amino acid HLA-A2 epitope (LLM) from human papilloma virus (HPV) E16 protein and cultured with an autologous HPVspecific CD8+ T cell line. As expected, in all conditions, the DCs efficiently presented HPV peptide to CD8+ T cells inducing IFN-γγ (FIG. 13a). However, dendritic cell presentation of HPV virus is dependent upon cross-presentation to the MHC class I pathway. Therefore we also tested the ability of untreated, microwave treated or cryotherapy KC-primed DCs to present human papilloma virus (HPV) E16 protein to an HLA matched HPV-specific CD8+ T cell line. Strikingly, only microwave treated KCs were able to prime DCs to enhance cross-presentation (FIG. 13b). To explore the potential mechanism of keratinocyte response to microwave therapy we confirmed up regulation of HSP-70 in response to microwave therapy of keratinocytes (FIG. 13c) and showed significant IL-6 induction in keratinocytes above that seen in cryotherapy treated cells (FIG. 13d). IRF1 and IRF4 are key regulators of dendritic cell activation and we confirmed that microwave therapy induced down regulation of IRF1 and up-regulation of IRF4 (FIG. 13e).

Discussion

This is the first study to investigate the potential efficacy of locally delivered microwaves in the treatment of cutaneous viral warts in vivo. We report a complete resolution rate of 75.9% recalcitrant plantar warts (average lesion duration of over 5 years). This compares very well with previous reports of plantar wart resolution for salicylic acid and or cryotherapy (23-33%)¹³. Whilst this study was a pilot phase, and did not include a control untreated arm, we believe the treatment effect to be significant.

For all novel therapies, adverse events are critical. In this study we did not identify a strong signal for adverse events with microwave therapy of cutaneous warts. As with current physical treatments for warts discomfort is expected for the patient. During the study patients typically reported that for a typical 5 second treatment that they endured moderate discomfort for approximately 2 seconds, which immediately diminished after the treatment had completed. In addition, it was commonly noted that discomfort was less with subsequent treatments. One male patient, withdrew from the study after one treatment, citing the pain of treatment as the reason. In the study design phase, preoperative use of topical anaesthetic cream was tested, but appeared to do little to mitigate the pain (unpublished data) and it was felt that the pain of local anaesthetic injection would exceed that normally experienced during a microwave treatment. Following microwave therapy, patients did not require dressings or special advice as microwave therapy utilised in this study did not cause a wound or ulcer in the skin, allowing the patient continue normal activity. The short microwave treatment time (5 s) offers a significant clinical advantage over current wart therapies such as cryotherapy and electro-surgery. Within 5 s, microwaves penetrate to a depth of over 3.5 mm at the energy levels adopted for the study¹⁴—possibly a greater depth than can be attained by cryosurgery or laser energy devices. Moreover, as microwaves travel in straight lines energy is deposited in alignment the device tip with little collateral spread, meaning minimal damage to surrounding tissue, as observed in this study. Microwaves induce dielectric heating. When water, as a polar molecule, is exposed to microwave energy, the molecule is excited and rotates attempting to align with the alternating electro-magnetic field. At microwave frequencies the molecule is unable to align fully with the continuously shifting field resulting in heat generation. Within tissues, this acts to rapidly elevate temperatures. This process rapidly changes cellular heat because it does not depend on tissue conduction. Microwave treatment produces no vapour or smoke unlike ablative lasers and electro-surgery, eliminating the need for air extraction systems due to the risk of spreading viral particles within the plume¹⁵.

Although, microwave therapy has been considered a tissue ablation tool, we saw minimal skin damage after treatment with 50 J, yet apparent good clinical response. Therefore we investigated whether there was evidence to support an induction of immunity by microwave therapy. The critical nature of CD8+ T cell immunity for host defence against HPV skin infection is well established and supported by the observation of increased prevalence of infection in immunosuppressed organ transplant recipients¹⁶, and that induction of protection from HPV vaccines is mediated by CD8+ T cells¹⁷. We show here, that microwave therapy of skin induces keratinocyte activation and cell death through apoptosis. However, at sub-apoptotic doses, microwave primed keratinocytes are able to signal to dendritic cells and enhance cross-presentation of HPV antigens to CD8+ lymphocytes which offers a potential explanation for the observed response rate in our clinical study. In vitro evidence suggests that this is likely to be mediated by cross-talk between microwave treated skin keratinocytes and dendritic cells, with resultant enhanced cross-presentation of HPV protein to CD8+ T cells. Microwave therapy also induced enhanced IL-6 synthesis from keratinocytes.

IL-6, is a pro-inflammatory mediator, important in anti-viral immunity which has been recently shown to induce rapid effector function in CD8+ cells¹⁸. Thus, IL-6 up-regulation may provide an important additional mechanism for microwave anti-viral immunity. IRFs have been shown to be central to the regulation of immune responses¹⁹⁻²¹. IRF4 is essential for differentiation of cytotoxic CD8+ T cells^{22 23}, but up-regulation in dendritic cells has also been shown to enhance CD4+ differentiation, thereby potentially enhancing both CD8+ immunity and T cell help following microwave treatment. IRF1 expression has been previously reported to be modulated by HPV infection, but different models have shown opposite outcomes^{24,25}. We show down-regulation of IRF1 in human skin in association with a microwave therapy which supports the proposal of IRF-1 as a therapeutic target in HPV infection²⁵.

This study is the first of its kind studying microwaves in the treatment of plantar warts in vivo. However, the authors acknowledge the limitations of the uncontrolled, non-randomised design. Despite the promising results shown here, studies with larger sample sizes are needed to assess the efficacy of this treatment and for infrequent but serious adverse events.

Additional Data

A surface-based microwave product was used on a patient diagnosed with terminal melanoma, the treatment granted on compassionate ethics. The clinically stable patient received a treatment dose of 8 GHz microwave energy at 10 W applied 3 times, with each application lasting 2 seconds and delivered via direct surface applicator contact on 2 lesions of the thigh. Treatment occurred on 2 visits, timed 1 month apart. The lymphatic glands had been removed from the leg prior to treatment. The existing TX was Pembrolizumab and Ipilimumab. The patient had a CT before and after the 2 treatments. The CT showed regression likely in the 2 sites. Physical palpitation in 1 of the sites suggested considerable regression. A follow-up CT approximately 8 months from previous showed further minimal improvement.

It will be understood that embodiments of the present invention have been described above purely by way of example, and modifications of detail can be made within the scope of the invention.

Each feature disclosed in the description, and (where appropriate) the claims and drawings may be provided independently or in any appropriate combination.

### REFERENCES

1. Cockayne S, Hewitt C, Hicks K, et al. Cryotherapy versus salicylic acid for the treatment of plantar warts (verrucae): a randomised controlled trial. BMJ 2011; 342:d3271.
2. Stern P L. Immune control of human papillomavirus (HPV) associated anogenital disease and potential for vaccination. Journal of clinical virology: the official publication of the Pan American Society for Clinical Virology 2005; 32 Suppl 1:S72-81.
3. Soong R S, Song L, Trieu J, et al. Toll-like receptor agonist imiquimod facilitates antigenspecific CD8+ T-cell accumulation in the genital tract leading to tumour control through IFNgamma. Clin Cancer Res 2014; 20:5456-67.
4. Edwards L, Ferenczy A, Eron L, et al. Self-administered topical 5% imiquimod cream for external anogenital warts. HPV Study Group. Human PapillomaVirus. Arch Dermatol 1998; 134:25-30.
5. Bristow I, Walker N. Pulsed Dye laser for the treatment of plantar warts—two case studies. Foot 1997; 7:229-30.
6. Kimura U, Takeuchi K, Kinoshita A, Takamori K, Suga Y. Long-pulsed 1064-nm neodymium:yttrium-aluminum-garnet laser treatment for refractory warts on hands and feet. The Journal of Dermatology 2014; 41:252-7.
7. Park H, Choi W. Pulsed dye laser treatment for viral warts: A study of 120 patients. Journal of Dermatology 2008; 35:491-8.
8. Tosti A, Piraccini B M. Warts of the Nail Unit: Surgical and Nonsurgical Approaches. Dermatol Surg 2001; 27:235-9.
9. Sterling J C, Gibbs S, Haque Hussain S S, Mohd Mustapa M F, Handfield-Jones S E. British Association of Dermatologists' guidelines for the management of cutaneous warts 2014. Br J Dermatol 2014; 171:696-712.
10. Lloyd D M, Lau K N, Welsh F, et al. International multicentre prospective study on microwave ablation of liver tumours: preliminary results. HPB: the official journal of the International Hepato Pancreato Biliary Association 2011; 13:579-85.
11. Polak M E, Thirdborough S M, Ung C Y, et al. Distinct molecular signature of human skin Langerhans cells denotes critical differences in cutaneous dendritic cell immune regulation. J Invest Dermatol 2014; 134:695-703.
12. Ressing M E, de Jong J H, Brandt R M, et al. Differential binding of viral peptides to HLA-A2 alleles. Implications for human papillomavirus type 16 E7 peptide-based vaccination against cervical carcinoma. European journal of immunology 1999; 29:1292-303.
13. Bruggink S C, Gussekloo J, Berger M Y, et al. Cryotherapy with liquid nitrogen versus topical salicylic acid application for cutaneous warts in primary care: randomised controlled trial. CMAJ 2010; 182:1624-30.
14. Emblation Medical Limited. Swift applicator instructions for use. Alloa, Scotland 2012.
15. Karsai S, Daschlein G. “Smoking guns”: Hazards generated by laser and electrocautery smoke. J Dtsch Dermatol Ges 2012; 10:633-6.
16. Tan H H, Goh C L. Viral infections affecting the skin in organ transplant recipients: epidemiology and current management strategies. Am J Clin Dermatol 2006; 7:13-29.
17. de Jong A, O'Neill T, Khan A Y, et al. Enhancement of human papillomavirus (HPV) type 16 E6 and E7-specific T-cell immunity in healthy volunteers through vaccination with TA-CIN, an HPV16 L2E7E6 fusion protein vaccine. Vaccine 2002; 20:3456-64.
18. Bottcher J P, Schanz O, Garbers C, et al. IL-6 trans-signaling-dependent rapid development of cytotoxic CD8+ T cell function. Cell reports 2014; 8:1318-27.
19. Schlitzer A, McGovern N, Teo P, et al. IRF4 transcription factor-dependent CD11b+ dendritic cells in human and mouse control mucosal IL-17 cytokine responses. Immunity 2013; 38:970-83.
20. Vander Lugt B, Khan A A, Hackney J A, et al. Transcriptional programming of dendritic cells for enhanced MHC class II antigen presentation. Nat Immunol 2013.
21. Tussiwand R, Lee W L, Murphy T L, et al. Compensatory dendritic cell development mediated by BATF-IRF interactions. Nature 2012; 490:502-7.
22. Huber M, Lohoff M. IRF4 at the crossroads of effector T-cell fate decision. European journal of immunology 2014; 44:1886-95.
23. Raczkowski F, Ritter J, Heesch K, et al. The transcription factor Interferon Regulatory Factor 4 is required for the generation of protective effector CD8+ T cells. Proc Natl Acad Sci USA 2013; 110:15019-24.
24. Park J S, Kim E J, Kwon H J, Hwang E S, Namkoong S E, Um S J. Inactivation of interferon regulatory factor-1 tumor suppressor protein by HPV E7 oncoprotein. Implication for the E7-mediated immune evasion mechanism in cervical carcinogenesis. J Biol Chem 2000; 275:6764-9.
25. Muto V, Stellacci E, Lamberti A G, et al. Human papillomavirus type 16 E5 protein induces expression of beta interferon through interferon regulatory factor 1 in human keratinocytes. Journal of virology 2011; 85:5070-80.

1. A method of raising an immune response in a subject said method comprising administering to a subject an immunostimulatory amount or dose of microwave energy.

2. The method of claim 1, wherein the method comprises administering repeated rounds of microwave energy.

3. The method of claim 1, wherein said melanoma is one or melanoma selected from the group consisting of:
(i) cutaneous melanoma
(ii) ocular melanoma;
(iii) melanoma of the vulva
(iv) melanoma of the vagina;
(v) melanoma of the rectum;
(vi) melanoma of the gastrointestinal tract;
(vii) superficial spreading melanoma;
(viii) nodular melanoma;
(ix) lentigo maligna melanoma;
(x) acral lentiginous melanoma; and
(xi) amelanotic melanoma.

4. The method of claim 1, wherein the microwave energy has a frequency of at least one of:—
between about 500 MHz and about 200 GHz;
between about 900 MHz and about 100 GHz;
between about 5 GHz to about 15 GHz.

5. The method of claim 1, wherein the microwave energy has a frequency of about 8 GHz.

6. The method of claim 1, wherein the microwave energy is applied at an energy of between about 1 J and about 200 J.

7. The method of claim 1, wherein the microwave energy is applied at an energy of about 5 J, about 10 J, about 50 J, about 100 J or about 200 J.

8. The method of claim 1, wherein the microwave energy is applied for anywhere between about 0.1 s and about 1 minute.

9. The method of claim 8, wherein the microwave energy is applied for about 5 s.

10. The method of claim 1, wherein the subject is exposed to one or more microwave energy doses.

11. The method of claim 1 wherein the dose or amount of microwave energy at least one of produces, induces, elevates an immune response and/or levels of heat shock factor HSF to stimulate production of a heat shock protein, in or near the tissue.

12. The method of claim 11, wherein the heat shock protein is selected from the group consisting of HSP90, HSP72, HSP70, HSP65. HSP60, HSP27, HSP16 and any another heat shock protein(s).

13. The method of claim 12, wherein the immune response is a cell and/or cytokine based immune response.

14. The method of claim 12, wherein the microwave energy promotes an association between the elevated heat shock protein and the melanoma so as to elicit an immune response against the melanoma.

15. The method of claim 1, being a method for treatment of a melanoma lesion, said method comprising delivering the therapeutically effective amount or dose of microwave energy to the melanoma lesion, wherein the microwave energy at least one of induces an immune response, cauterises, coagulates, shrinks, blocks, ablates, damages, irritates, inflames, otherwise interferes with the normal operation of the capillaries supplying blood to the lesion.

16. The method of claim 1, being a method for the treatment or prevention of a melanoma lesion, said method comprising delivering a therapeutically effective and/or immunostimulatory amount or dose of microwave energy to the melanoma lesion or tissue susceptible to the formation of a melanoma lesion, wherein the microwave energy induces an immune response within the lesion thus exposing antigenic sites further stimulating an immune response.

17-23. (canceled)

24. The method of claim 1, wherein the microwave energy is applied to the site of a melanoma or melanoma lesion.

25. The method of claim 1, wherein the microwave energy is applied directly to a melanoma lesion to be treated.

26. The method of claim 1, wherein the immune response is local to the site of a melanoma.

27. The method of claim 1, wherein the immune response is a protective or at least partially protective immune response.

28. The method of claim 27, wherein the protective or partially protective immune response, is effective against infection melanoma or melanoma lesion.

29. The method of claim 1, wherein the immune response is a cell and/or cytokine based immune response.

30. The method of claim 1, wherein the immune response comprises one of more responses selected from the group consisting of:
(i) an anti-melanoma immune response;
(ii) a cytokine based response;
(iii) an immune cell response; and
(iv) enhanced melanoma antigen presentation.

31-39. (canceled)

40. The method of claim 1, wherein:
the administering of the therapeutically effective amount or dose of microwave energy comprises using a delivery system to administer the therapeutically effective amount or dose of microwave energy via a microwave applicator to tissue of the subject that has melanoma or is susceptible/predisposed to the development of melanoma.

41. The method of claim 40, wherein the method comprises electrically matching the microwave applicator to the tissue that has said melanoma, based on the tissue that has said melanoma having a higher dielectric constant than a dielectric constant that said tissue would have if said tissue did not have said melanoma, such that the microwave applicator is better matched to the tissue that has said melanoma than it would have been if said tissue did not have said melanoma or melanoma lesion.

42. The method of claim 41, wherein the microwave energy is selected such as to stimulate a localised immune response at the melanoma and the administering of the therapeutically effective amount or dose of microwave energy to the melanoma comprises repeatedly applying the microwave energy in a pulsed manner thus providing repeated rounds of localised hyperthermia at the melanoma and repeated localised stimulation of the immune response at the melanoma.

43. A method of treating or preventing melanoma, said method comprising administering to a subject having melanoma or predisposed/susceptible to developing melanoma, a therapeutically effective amount or dose of microwave energy and one or more additional treatments selected from the group consisting of:
(i) checkpoint inhibitors;
(ii) vaccines;
(ii) chemotherapy; and/or
(iii) radiotherapy.

44. The method of claim 43, wherein the checkpoint inhibitor is selected from the group consisting of:
(i) a PD-L1 inhibitor
(ii) a PD-1 inhibitor
(iii) a CTLA-4 inhibitor
(iv) Pembrolizumab (PD-1 inhibitor);
(v) Nivolumab (PD-1 inhibitor);
(vi) Atezolizumab (PD-L1 inhibitor);
(vii) Avelumab (PD-L1 inhibitor);
(viii) Durvalumab (PD-L1 inhibitor); and
(ix) Ipilimumab (CTLA-4 inhibitor).

45. The method of claim 43, wherein the chemotherapy exploits one or more chemotherapeutic agents selected from the group consisting
(i) Dacarbazine (also called DTIC)
(ii) Temozolomide
(iii) Nab-paclitaxel
(iv) Paclitaxel
(v) Cisplatin
(vi) Carboplatin
(vii) Vinblastine

46. The method of claim 43, wherein the checkpoint inhibitor is administered together with or before and/or after the microwave based treatment.

47. The method of claim 43, wherein the vaccine is administered together with or before and/or after the microwave based treatment.

48. The method of claim 43, wherein the chemotherapy is administered together with or before and/or after the microwave based treatment.

49. The method of claim 43, wherein the radiotherapy is administered together with or before and/or after the microwave based treatment.

50. The method of claim 43, wherein the microwave based treatment is intended to promote prolonged down-regulation of IL-10, IL-4 and/or other anti-inflammatory cytokines, and/or other negative regulation factors in the anti-tumour immune system response, to enhance the durability of action or use of checkpoint inhibitors with or before and/or after the microwave based treatment.

51. The method of claim 1, wherein the subject has, is predisposed or susceptible to melanoma.
